(19) United States
(12) Patent Application Publication
(10) Pub. No.: US 2009/0018097 A1
(43) Pub. Date: Jan. 15, 2009

(54) MODIFICATION OF DOUBLE-STRANDED RIBONUCLEIC ACID MOLECULES

(75) Inventors: Steven C. Quay, Woodinville, WA (US); Kunyuan Cui, Bothell, WA (US); Paul H. Johnson, Snohomish, WA (US); Lishan Chen, Bellevue, WA (US); Mohammad Ahmadian, Bothell, WA (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: MDRNA, INC, Bothell, WA (US)

(21) Appl. No.: 12/065,604

(22) PCT Filed: May 25, 2006
(86) PCT No.: PCT/US06/20627

§ 371 Date: Sep. 4, 2008

Related U.S. Application Data
(60) Provisional application No. 60/720,072, filed on Sep. 23, 2005.

(51) Int. Cl. A61K 31/713; C07H 21/02; C12N 5/10; A61P 35/00

(52) U.S. Cl. 514/44; 536/24.5; 435/375

(57) ABSTRACT

A double-stranded RNA (dsRNA) molecule comprising between about 15 base pairs and about 40 base pairs, wherein at least one ribonucleotide of the dsRNA is a 5′-methyl-pyrimidine and/or at least one 2′-O-methyl ribonucleotide, and a method of improving ribonuclease stability, reducing off-target effects of a double stranded siRNA molecule, or of reducing interferon responsiveness of a double stranded siRNA molecule using such dsRNA.

### TECHNICAL FIELD

This invention relates generally to the field of double stranded (ds) RNA preparation, particularly, modification of the dsRNA to improve stability, maximize target RNA knockdown efficacy, minimize “off-target” effect and maximize capture of target RNA variants. The invention further relates to the treatment of disorders by means of RNA interference (RNAi).

### BACKGROUND ART

RNA interference refers to the process of sequence-specific post-transcriptional gene silencing in animals mediated by short interfering RNAs (siRNAs). See Fire et al., Nature, 391:806 (1998) and Hamilton et al., Science, 286:950-951 (1999). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response though a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Hamilton et al., supra; Berstein et al., Nature, 409:363 (2001)). Short interfering RNAs derived from dicer activity are typically about 21 to about 23 nucleotides in length and comprise about 19 base pair duplexes (Hamilton et al., supra; Elbashir et al., Genes Dev., 15:188 (2001)). Dicer has also been implicated in the excision of 21- and 22-nucleotide small temporal RNAs (stRNAs) from precursor RNA of conserved structure that are implicated in translational control (Hutvagner et al., Science, 293:834 (2001)). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188 (2001)).

RNAi has been studied in a variety of systems (Fire et al., Nature, 391:806 (1998)) were the first to observe RNAi in C. elegans. Bahramian and Zarbl, Molecular and Cellular Biology, 19:274-283 (1999) and Wianny and Goetz, 1999, Nature Cell Biol., 2, 70, describe RNAi mediated by dsRNA in mammalian systems. Hammond et al., Nature, 404:293 (2000), describe RNAi in Drosophila cells transfected with dsRNA. Elbashir et al., Nature, 411:494 (2001), describe RNAi induced by introduction of duplexes of synthetic 21-nucleotide RNAs in cultured mammalian cells including human embryonic kidney and HeLa cells. Recent work in Drosophila embryonic lysates (Elbashir et al., EMBO J, 20:6877 (2001)) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., EMBO J, 20:6877 (2001)). Other studies have indicated that a 5′-phosphate on the target-complementary strand of a siRNA duplex is required for siRNA activity and that ATP is utilized to maintain the 5′-phosphate moiety on the siRNA (Nykanen et al., Cell, 107:309 (2001)).

Recent developments in the areas of gene therapy, antisense therapy and RNA interference therapy have created a need to develop efficient means of introducing nucleic acids into cells. Unfortunately, existing techniques for delivering nucleic acids to cells are limited by instability of the nucleic acids, poor efficiency and/or high toxicity of the delivery reagents.

Thus, there is a need to provide for methods and compositions for effectively delivering double-stranded nucleic acids to cells to produce an effective therapy especially for delivering siRNAs for RNA interference therapy.

### BRIEF DESCRIPTION OF DRAWINGS

FIG. 1 is an SDS PAGE gel showing the results of the stability studies of Example 3, in which the stable siRNA construct in which all of the uridines are changed to 5-methyluridine ribothymidine.

FIG. 2 shows the knockdown activities for LC20-MD3, MD-6, MD-8, MD-15, MD-17, MD-18 and MD19. The solid bars represent an siRNA concentration of 0.16 nM, the bars with horizontal stripes represent an siRNA concentration of 0.8 nM and the bars with black and white diamonds represent an siRNA concentration of 4 nM. Knockdown activities were normalized to the Qneg control siRNA and presented as a percentage of the Qneg control (i.e., Qneg represented 100% or “normal” gene expression levels). Thus, a smaller percentage indicates a greater knockdown effect.

FIG. 3 shows the degradation time-course and the degradants for the non-modified siRNA duplex at time zero (non-incubated) and incubated with human plasma for 1 minute, 60 minutes and 240 minutes.

FIG. 4 shows the degradation time-course and the degradants for the modified siRNA duplex at time zero (non-incubated) and incubated with human plasma for 1 minute, 60 minutes and 240 minutes.

FIG. 5 summarizes the degradation profiles for both the sense and anti-sense strands of the non-modified (native) and modified siRNA duplexes.

### MODES FOR CARRYING OUT THE INVENTION

The instant invention provides novel short interfering RNAs (siRNAs), having improved stability for example to exonuclease degradation, in eukaryotic cells and other physiological environments, including plant and animal tissues and fluid compartments. Also provided herein are methods of making chemically modified siRNAs having improved stability, as well as compositions and methods for inhibiting the expression of a target gene using a chemically modified siRNA. Further provided are modified siRNAs that mediate gene silencing while reducing or preventing off-target effects, including off target interference with non-target gene expression, and minimizing or preventing activation of an interferon response in target cells. The present invention also relates to the targeted delivery of siRNA that are capable of mediating RNAi against genes, and variants thereof, wherein the siRNA comprise one or more universal-binding nucleotide.

In one embodiment of the invention, the siRNAs comprise one or more, multiply-modified ribonucleotides according to Formula I, below:

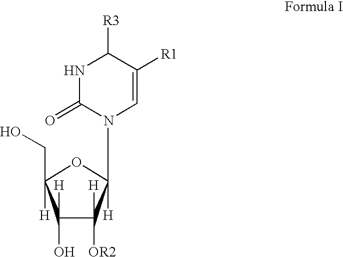

wherein R₃ comprises a carbonyl or amino group (consistent with a pyrimidine structure of uracil, or cytosine, respectively), R₁ comprises a chemically modified or substituted group at a C-5 position of the pyrimidine, and R₂ comprises a chemically modified or substituted group at a 2′ position of the ribose.

Within exemplary embodiments of the invention, the compounds of Formula I are provided wherein R₁ comprises a 5-alkyl substituted pyrimidine, for example a 5-alkyl uridine, or a 5-alkyl cytidine. In more detailed embodiments, the 5-alkyl uridine, or 5-alkyl cytidine is a 5-methyl uridine or 5-methyl cytidine. In alternate embodiments, the 5-substituted pyrimidine is engineered to include, at the C-5 position of the pyrimidine, a different chemical modification or substitution, for example a chemical substituent selected from a halogen, hydroxy, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, carbonyl, alkanoylamino, carbamoyl, carbamyl, carbonylamino, alkylsulfonylamino, or heterocyclo group.

As generally illustrated in Formula I above, the modified ribose of a multiply-modified ribonucleotide according to the invention will incorporate a separate modification independent from the modification of the pyrimidine. In exemplary embodiments, compounds of Formula I are provided wherein R₂ of the ribose comprises a 2′-alkyl substitution, for example a 2′-O-methyl substitution. In alternate embodiments, the modified or substituted ribose is engineered to include a different chemical modification or substitution at the 2′ position, for example a 2′ substituent selected from a halogen, hydroxy, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, carbonyl, alkanoylamino, carbamoyl, carbamyl, carbonylamino, alkylsulfonylamino, or heterocyclo group.

In certain exemplary embodiments of the invention, a multiply-modified ribonucleotide incorporated into a modified siRNA comprises a 5-R₁-pyrimidine and 2′-O—R₂ modified ribose. In exemplary embodiments, R₁ and/or R₂ is an alkyl. In more detailed embodiments, both R₁ and R₂ are alkyls. In yet more detailed embodiments, R₁ and/or R₂ is a methyl, or both R₁ and R₂ are methyls (i.e., the modified ribonucleotide is a 5-methylpyrimidine, such as 5-methyl-uridine (ribothymidine, or rT), having a ribose comprising a 2′-O-methyl modification)).

In another exemplary of the invention, the siRNAs comprise at least one multiply-modified ribonucleotide according to Formula I. In certain embodiments, siRNAs of the invention comprise two or more multiply-modified ribonucleotide according to Formula I. When two or more modified ribonucleotides are present, each modified ribonucleotide can be independently modified to have the same, or different, modification or substitution at R₁, R₂, and R₃. Thus, two or more modified ribonucleotides can each be independently selected wherein R₃ is a carbonyl group and the subject pyrimidine(s) is/are a cytosine, or wherein R₃ is an amino group and the subject pyrimidine(s) is/are a cytosine. Likewise, two or more modified ribonucleotides can each be independently selected wherein R₁ comprises any chemical modification or substitution as contemplated herein, for example an alkyl (e.g., methyl), halogen, hydroxy, alkoxy, nitro, amino, trifluoromethyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, carbonyl, alkanoylamino, carbamoyl, carbamyl, carbonylamino, alkylsulfonylamino, or heterocyclo group.

Within additional aspects of the invention, siRNAs are constructed to include one or more multiply-modified ribonucleotide(s) according to Formula I distributed on one strand, or on both strands, of the siRNA. In exemplary embodiments, the modified ribonucleotide(s) is/are incorporated at one or both of the 3′ and 5′ termini of the strand or strands bearing the modified ribonucleotide(s). Thus, exemplary siRNAs according to this aspect of the invention can have one or more multiply-modified ribonucleotide(s) according to Formula I located at either or both of the 3′ and 5′ termini of the sense strand, and/or, at either or both of the 3′ and 5′ termini of the anti-sense strand.

Within more detailed embodiments, both the sense and anti-sense strands of the siRNA bear at least one multiply-modified ribonucleotide according to Formula I. Within this aspect of the invention, exemplary embodiments are provided wherein the siRNA has a multiply-modified ribonucleotide according to Formula I at the 5′ termini of both the sense and anti-sense strands. Other exemplary embodiments are provided wherein the siRNA has a multiply-modified ribonucleotide according to Formula I at the 3′ termini of both the sense and anti-sense strands. Still other exemplary embodiments are provided wherein the siRNA has a multiply-modified ribonucleotide according to Formula I at the 3′ and 5′ termini of both the sense and anti-sense strands.

In other detailed embodiments, one or more multiply-modified ribonucleotide(s) according to Formula I can be located at any ribonucleotide position, or any combination of ribonucleotide positions, on either or both of the sense and anti-sense strands of a modified siRNA, including at one or more multiple terminal positions as noted above, and/or at any one or combination of multiple non-terminal (“internal”) position(s). In this regard, each of the sense and anti-sense strands can incorporate 1, 2, 3, 4, 5, 6, or more of the multiply-modified ribonucleotides.

Where two or more multiply-modified ribonucleotides are incorporated within an siRNA of the invention, often at least one of the modified ribonucleotides will be at a 3′ or 5′ end of one or both strands, and in certain embodiments at least one of the modified ribonucleotides will be at a 5′ end of one or both strands. Typically, the multiply modified ribonucleotides are located at a position corresponding to a position of a pyrimidine in an non-modified siRNA that is constructed as a homologous sequence for targeting a cognate mRNA, as described herein below.

Incorporation of a multiply-modified polynucleotide into an siRNA according to the invention will often increase resistance of the siRNA to enzymatic degradation, particularly exonucleolytic degradation, including 5′ exonucleolytic and/or 3′ exonucleolytic degradation. As such, the siRNAs described herein will exhibit significant resistance to enzymatic degradation compared to a corresponding, non-modified siRNA, and will thereby possess greater stability, increased half life, and greater bioavailability in physiological environments (e.g., when introduced into a eukaryotic target cell). In certain embodiments, selected modifications of siRNAs according to the instant invention employ novel combinations of individual siRNA pyrimidine and ribose modifications described in U.S. patent application Ser. No. 10/925,314, filed Aug. 24, 2004 (and the priority provisional filing to this application, U.S. Provisional Application No. 60/497,740 filed Aug. 25, 2003); and U.S. application Ser. No. 11/219,625, filed Sep. 2, 2005 and U.S. application Ser. No. 11/219,582 filed Sep. 2, 2005, each of which disclosures is incorporated herein by reference in its entirety. In addition to increasing resistance of the modified siRNAs to exonucleolytic degradation, the incorporation of one or more multiply-modified ribonucleotide(s) according to Formula I will render siRNAs more resistant to other enzymatic and/or chemical degradation processes, and thus more stable and bioavailable than otherwise identical siRNAs that do not include the modified ribonucleotide(s). In related aspects of the invention, siRNA modifications described herein will often improve stability of a modified siRNA for use within research, diagnostic and treatment methods wherein the modified siRNA is contacted with a biological sample, for example, a mammalian cell, intracellular compartment, serum or other extracellular fluid, tissue, or other in vitro or in vivo physiological compartment or environment. In one embodiment, diagnosis is performed on an isolated biological sample. In another embodiment, the diagnostic method is performed in vitro. In a further embodiment, the diagnostic method is not performed (directly) on a human or animal body.

In addition to increasing stability of modified siRNAs, incorporation of one or more multiply-modified polynucleotides according to Formula I in an siRNA designed for gene silencing will yield additional desired functional results, including increasing a melting point of a modified siRNA compared to a corresponding, non-modified siRNA. By thus increasing an siRNA melting point, the subject modifications will often block or reduce the occurrence or extent of partial dehybridization of the modified siRNA (that would ordinarily occur and render the non-modified siRNA more vulnerable to degradation by certain exonucleases), thereby increasing the stability of the modified siRNA.

In another aspect of the invention, chemical modifications of siRNAs described herein will reduce “off-target effects” of the modified siRNA molecules when they are contacted with a biological sample (e.g., when introduced into a target eukaryotic cell having specific, and non-specific mRNA species present as potential specific and non-specific targets). In related embodiments, modified siRNAs according to the invention are employed in methods of gene silencing, wherein the modified siRNAs exhibit reduced or eliminated off target effects compared to a corresponding, non-modified siRNA, e.g., as determined by non-specific activation of genes in addition to a target (i.e., homologous or cognate) gene in a cell or other biological sample to which the modified siRNA is exposed under conditions that allow for gene silencing activity to be detected.

In yet another aspect of the invention, the siRNA modifications described herein will reduce interferon activation by the siRNA molecule when the siRNA is contacted with a biological sample, e.g., when introduced into a eukaryotic cell.

In yet another aspect, the invention provides methods for inhibiting expression of a target gene in a eukaryotic cell. The method includes introducing a modified siRNA of the invention into the cell, and maintaining the cell for a time sufficient to allow the siRNA to mediate downregulation of gene expression, which will typically include degradation of a mRNA transcript of a targeted gene. In the case of mammalian subjects, those subjects amenable for treatment using the compositions and methods of the invention will include human and other mammalian subjects suffering from one or more diseases or conditions mediated, at least in part by overexpression of a targeted gene. In exemplary embodiments, the methods and compositions of the invention are employed to treat a disease or condition mediated by overexpression of one or more target genes/proteins, for example a cellular proliferative disorder, differentiative disorder, disorder associated with bone metabolism, immune disorder, hematopoietic disorder, cardiovascular disorder, liver disorder, viral disease, or metabolic disorder.

Within additional detailed aspects of the invention, modifications of siRNAs to incorporate a multiply-modified ribonucleotide of Formula I at the 3′ and/or 5′ end of one or both strands of the siRNA, with few or limited modified ribonucleotides present at internal positions in the siRNA, yields a more stable and functional siRNA (i.e., in comparison to a sequence-identical but non-modified siRNA, or an siRNA incorporating several modified ribonucleotides according to the invention internally. Accordingly, in certain embodiments the invention, multiply-modified ribonucleotides according to Formula I are concentrated at the 3′ and/or 5′ end(s) of the siRNA. Often, incorporation of the modified ribonucleotide(s) is limited to the 5′ ends of both the sense and anti-sense strands, alternatively to the 3′ ends of both the sense and anti-sense strands, and in other alternate embodiments to the 3′ and 5′ ends of both strands. Typically, fewer than 10, often fewer than 8, more often fewer than 6, and usually less then 2-4 multiply-modified ribonucleotides are incorporated internally within a sense or anti-sense strand, or among both strands collectively, in the modified siRNA.

siRNAs of the invention typically comprise a double stranded RNA (dsRNA) molecule comprising a sense strand that is homologous to a sequence of a target gene and an anti-sense strand that is complementary to said sense strand. In exemplary embodiments, at least one strand of the siRNA incorporates one or more pyrimidines modified according to Formula I (e.g., wherein the pyrimidine is replaced by a ribothymidine, and the ribose is modified to incorporate a 2′-O-methyl substitution). These and other multiple modifications according to Formula I can be introduced into one or more pyrimidines, or into any combination and up to all pyrimidines present in one or both strands of the siRNA.

Within certain aspects, the present invention provides siRNA, and compositions comprising one or more siRNA, wherein at least one of the siRNA comprises one or more universal-binding nucleotide(s) in the first, second and/or third position in the anti-codon of the anti-sense strand of the siRNA duplex and wherein said siRNA is capable of specifically binding to a RNA, such as an RNA expressed by a target virus. In cases wherein the sequence of the target virus RNA includes one or more single nucleotide substitution, the universal-binding nucleotide comprising siRNA retains its capacity for specifically binding to the target virus RNA thereby mediating gene silencing and, as a consequence, overcoming escape of the target virus to siRNA-mediated gene silencing.

Non-limiting examples of universal-binding nucleotides that may be suitably employed in the compositions and methods disclosed herein include inosine, 1-β-D-ribofuranosyl-5-nitroindole, and 1-β-D-ribofuranosyl-3-nitropyrrole. For the purpose of the present invention, a universal-binding nucleotide is a nucleotide that can form a hydrogen bonded nucleotide pair with more than one nucleotide type.

Non-limiting examples for the above compositions includes modifying the anti-codons for tyrosine (AUA) or phenylalanine (AAA or GAA), cysteine (ACA or GCA), histidine (AUG or GUG), asparagine (AUU or GUU), isoleucine (UAU) and aspartate (AUC or GUC) within the anti-codon of the anti-sense strand of the siRNA molecule.

For example, within certain embodiments, the isoleucine anti-codon UAU, for which AUA is the cognate codon, may be modified such that the third-position uracil (U) nucleotide is substituted with the universal-binding nucleotide inosine (I) to create the anti-codon UAI. Inosine is a universal-binding nucleotide that can nucleotide-pair with an adenine (A), uracil (U), and cytosine (C) nucleotide, but not guanine (G). This modified anti-codon UAI increases the specific-binding capacity of the siRNA molecule and thus permits the siRNA to pair with mRNAs having any one of AUA, UUA, and CUA in the corresponding position of the coding strand thereby expanding the number of available RNA degradation targets to which the siRNA may specifically bind.

Alternatively, the anti-codon AUA may also or alternatively be modified by substituting a universal-binding nucleotide in the third or second position of the anti-codon such that the anti-codon(s) represented by UAI (third position substitution) or UIU (second position substitution) to generate siRNA that are capable of specifically binding to AUA, CUA and UUA and AAA, ACA and AUA.

Typically, siRNA of the present invention comprise between about 15 base-pairs and about 40 base-pairs; more typically, between about 18 and 35 base-pairs; still more typically between about 20 and 30 base-pairs; and most typically either 21, 22, 23, 24, 25, 26, 27, 28, or 29 nucleotides and may comprise a single-strand overhang of between 0 nucleotides and 5 nucleotides, most typically, the single-strand 3′ overhang is 1, 2, 3, or 4 nucleotides. Regardless of the precise length of the siRNA duplex and optional overhanging sequence, the siRNA duplex will comprise at least one or more universal-binding nucleotide, wherein the at least one or more universal-binding nucleotide may be selected from the group consisting of inosine, 1-β-D-ribofuranosyl-5-nitroindole, and 1-β-D-ribofuranosyl-3-nitropyrrole.

Typically, siRNA disclosed herein will include between about 1 universal-binding nucleotide and about 10 universal-binding nucleotides. For example, siRNA of the present invention may include 1, 2, 3, 4, 5, 6, 7, 8, 9, or 10 universal-binding nucleotides. Within certain aspect, the presently disclosed siRNA may comprise a sense strand that is homologous to a sequence of a target gene and an anti-sense strand that is complementary to the sense strand, with the proviso that at least one nucleotide of the anti-sense strand of the otherwise complementary siRNA duplex is replaced by one or more universal-binding nucleotide.

It will be understood that, regardless of the position at which the one or more universal-binding nucleotide is substituted, the siRNA molecule is capable of binding to a target gene and one or more variant(s) thereof thereby facilitating the degradation of the target gene and/or variant thereof via a RISC complex. Thus, the siRNA of the present invention are suitable for introduction into cells to mediate targeted post-transcriptional gene silencing of a target gene and/or variants thereof. When an siRNA is inserted into a cell, the siRNA duplex is then unwound, and the anti-sense strand of the duplex is loaded into an assembly of proteins to form the RNA-induced silencing complex (RISC).

Within the silencing complex, the siRNA molecule is positioned so that RNAs can bump into it. The RISC will encounter thousands of different RNAs that are in a typical cell at any given moment. But the siRNA of the RISC will adhere well only to an RNA that closely complements its own nucleotide sequence. So unlike an interferon response to a viral infection, the silencing complex is highly selective in choosing its target RNAs.

When a matched RNA finally docks onto the siRNA, an enzyme know as dicer cuts the captured RNA strand in two. The RISC then releases the two pieces of the RNA (now rendered incapable of directing protein synthesis) and moves on. The RISC itself stays intact capable of finding and cleaving another RNA.

One embodiment of the present invention is comprised of nanoparticles of double-stranded RNA less than 100 nanometers (nm). More, specifically, the double-stranded RNA is less than about 30 base-pairs in length, preferably 20-25 nucleotide base-pairs in length.

Compositions and methods disclosed herein are useful in the treatment of a wide variety viral infections caused by target viruses including, but not limited to, a retrovirus, such as human immunodeficiency virus (HIV), as well as respiratory viruses, such as human respiratory syncytial virus, human metapneumovirus, human parainfluenza virus 1, human parainfluenza virus 2, human parainfluenza virus 3, human parainfluenza virus 4a, human parainfluenza virus 4b, influenza A virus, influenza B virus, rhinovirus and influenza C virus.

Within additional aspects of the invention, modifications of siRNAs comprise the incorporation of one or more multiply-modified ribonucleotides of Formula I and one or more universal-binding nucleotide(s).

The present invention also features a method for preparing the claimed dsRNA nanoparticles. A first solution containing melamine derivatives is dissolved in an organic solvent such as dimethyl sulfoxide, or dimethyl formamide to which an acid such as HCl has been added. The concentration of HCl would be about 3.3 moles of HCl for every mole of the melamine derivative. The first solution is then mixed with a second solution, which includes a nucleic acid dissolved or suspended in a polar or hydrophilic solvent (e.g., an aqueous buffer solution containing, for instance, ethylenediaminetraacetic acid (EDTA), or tris(hydroxymethyl)aminomethane (TRIS), or combinations thereof. The mixture forms a first emulsion. The mixing can be done using any standard technique such as, for example sonication, vortexing, or in a microfluidizer. This causes complexing of the nucleic acids with the melamine derivative forming a trimeric nucleic acid complex. While not being bound to theory or mechanism, it is believed that three nucleic acids are complexed in a circular fashion about one melamine derivative moiety, and that a number of the melamine derivative moieties can be complexed with the three nucleic acid molecules depending on the size of the number of nucleotides that the nucleic acid has. The concentration should be at least 1 to 7 moles of the melamine derivative for every mole of a double stranded nucleic acid having 20 nucleotide pairs, more if the ds nucleic acid is larger. The resultant nucleic acid particles can be purified and the organic solvent removed using size-exclusion chromatography or dialysis or both.

The complexed nucleic acid nanoparticles can then be mixed with an aqueous solution containing either polyarginine, a Gln-Asn polymer, or both in an aqueous solution. The preferred molecular weight of each polymer is 5000-15,000 Daltons. This forms a solution containing nanoparticles of nucleic acid complexed with the melamine derivative and the polyarginine and the Gln-Asn polymers. The mixing steps are carried out in a manner that minimizes shearing of the nucleic acid while producing nanoparticles on average smaller than 200 nanometers in diameter. While not being bound by theory of mechanism, it is believed that the polyarginine complexes with the negative charge of the phosphate groups within the minor groove of the nucleic acid, and the polyarginine wraps around the trimeric nucleic acid complex. At either terminus of the polyarginine other moieties, such as the TAT polypeptide, mannose or galactose, can be covalently bound to the polymer to direct binding of the nucleic acid complex to specific tissues, such as to the liver when galactose is used. While not being bound to theory, it is believed that the Gln-Asn polymer complexes with the nucleic acid complex within the major groove of the nucleic acid through hydrogen bonding with the bases of the nucleic acid. The polyarginine and the Gln-Asn polymer should be present at a concentration of 2 moles per every mole of nucleic acid having 20 base pairs. The concentration should be increased proportionally for a nucleic acid having more than 20 base pairs. So perhaps, if the nucleic acid has 25 base pairs, the concentration of the polymers should be 2.5-3 moles per mole of ds nucleic acid. An example of is a polypeptide operatively linked to an N-terminal protein transduction domain from HIV TAT. The HIV TAT construct for use in such a protein is described in detail in Vocero-Akbani et al., Nature Med., 5:23-33 (1999). See also, United States Patent Application No. 20040132161, published on Jul. 8, 2004.

The resultant nanoparticles can be purified by standard means such as size exclusion chromatography followed by dialysis. The purified complexed nanoparticles can then be lyophilized using techniques well known in the art.

A preferred embodiment of the present invention is comprised of nanoparticles of double-stranded RNA less than 100 nanometers (nm). More, specifically, the double-stranded RNA is less than about 30 nucleotide pairs in length, preferably 20-25 nucleotide base pairs in length. More specifically, the present invention is comprised of a double-stranded RNA complex wherein two or more double-stranded

In a preferred embodiment, the ribose uracils of the siRNA are replaced with ribose thymine. In fact it has been surprisingly discovered that the stability of double-stranded RNA is greatly increased and is less susceptible to degradation by Rnases when all of the ribose uracils are change to ribose thymine in both the sense and anti-sense strands of the RNA. Thus a preferred siRNA is a double-stranded RNA having 15-30 bases pairs wherein all of the ribose uracils that would normally be present have been changed to a 5-alkyluridine such as ribothymidine (rT) (5-methyluridine). Alternatively, some of the uracils can be changed so that only those ribose uracils present in the sense strand are changed to ribothymidine, or in the alternative, only those ribose uracils present in the antisense strand are changed to ribothymidine. Examples 2 and 3 illustrate this aspect of the invention.

For example a stable siNA duplex of the present invention which would target the mRNA of the VEGF receptor 1 (see SEQ ID NO:2000 of United States Patent Application Publication No. 2004/01381 published Jul. 15, 2004 would be:

(SEQ ID NO:9)

G.C.A.rT.rT.rT.G.G.C.A.rT.A.A.G.A.A.A.rTdTdT

(SEQ ID NO:10)

A.rT.rT.rTrT.C.rT.rT.A.rT.G.C.C.A.A.A.rT.C.dT.dT

An siNA duplex of the present invention, which would target the RNA of Hepatitis B virus and target a subsequence of the HBV RNA would be:

(SEQ ID NO:11)

C.C.rT.G.C.rT.G.C.rT.A.rT.G.C.C.rT.C.A.rT.C.dT.dT

(SEQ ID NO:12)

G.A.rT.G.A.G.G.C,A.rT.A.G.C.A.G.C.A.G.G.dTdT

See United States Patent Application Publication No. 2003/0206887 published Nov. 6, 2003.

An siNA duplex of the present invention which would target RNA of the human immunodeficiency virus (HIV) would be:

(SEQ ID NO:13)

rT.rT.rT.G.G.A.A.A.G.G.A.C.C.A.G.C.A.A.A.dT.dT

(SEQ ID NO:14)

rT.rT.rT.G.C.rT.G.G.rT.C.C.rTrT.rT.C.C.A.A.A.dT.dT

See United States Patent Application Publication No. 2003/0175950 published Sep. 18, 2003.

An siNA duplex of the present invention which would target the mRNA of human tumor necrosis factor-alpha (TNF-α) would be:

(SEQ ID NO:15)

C.A.C.C.C.rT.G.A.C.A.A.G.C.rT.G.C.C.A.G.dT.dT

(SEQ ID NO:16)

C.rT.G.G.C.A.G.C.rT.rT.G.rT.C.A.G.G.G.rT.G.dT.dT

Another siNA targeted against the TNF-α mRNA would be:

(SEQ ID NO:17)

rT.G.C.A.C.rT.rT.rT.G.G.A.G.rT.G.A.rT.C.G.G.dT.dT

(SEQ ID NO:18)

C.C.G.A.rT.C.A.C.rT.C.C.A.A.A.G.rT.G.C.A.dT.dT

An siNA duplex of the present invention targeted against the TNF-α-receptor 1A mRNA would be:

(SEQ ID NO:19)

G.A.G.rT.C.C.C.G.G.G.A.A.G.C.C.C.C.A.G.dT.dT

(SEQ ID NO:20)

C.rT.G.G.G.G.C.rTrT.C.C.C.G.G.G.A.C.rT.C.dT.dT

Another siNA duplex of the present invention targeted against the TNF-α-receptor 1A mRNA would be:

(SEQ ID NO:21)

A.A.A.G.G.A.A.C.C.rT.A.C.rT.rT.G.rT.A.C.A.dT.dT

(SEQ ID NO:22)

rT.G.rT.A.C.A.A.G.rT.A.G.G.rT.rT.C.C.rT.rT.rT.dT.
dT

See International Patent Application Publication No. WO 03/070897. ‘RNA Interference Mediated Inhibition of TNF and TNF Receptor Superfamily Gene Expression Using Short Interfering Nucleic Acid (siNA)’. These would be useful in treating TNF-α associated diseases as rheumatoid arthritis.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

In another aspect, the invention provides mammalian cells containing one or more siNA molecules of this invention. The one or more siNA molecules can independently be targeted to the same or different sites.

By “RNA” is meant a molecule comprising at least one ribonucleotide residue. By “ribonucleotide” is meant a nucleotide with a hydroxyl group at the 2′ position of a .beta.-D-ribo-furanose moiety. The term RNA includes, for example, double-stranded (ds) RNAs; single-stranded RNAs; and isolated RNAs such as partially purified RNA, essentially pure RNA, synthetic RNA, recombinantly produced RNA, as well as altered RNA that differ from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “sense region” is meant a nucleotide sequence of a siRNA molecule having complementarity to an anti-sense region of the siRNA molecule. In addition, the sense region of a siRNA molecule can comprise a nucleic acid sequence having homology with a target nucleic acid sequence.

By “anti-sense region” is meant a nucleotide sequence of a siRNA molecule having complementarity to a target nucleic acid sequence. In addition, the anti-sense region of a siRNA molecule can optionally comprise a nucleic acid sequence having complementarity to a sense region of the siRNA molecule.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, 2001, Nucleic Acids Research, 29:2437-2447).

The term “universal-binding nucleotide” as used herein refers to a nucleotide analog that is capable of forming a base-pairs with each of the natural DNA/RNA nucleotides with little discrimination between them. Non-limiting examples of universal-binding nucleotides include inosine, 1-β-D-ribofuranosyl-5-nitroindole, and/or 1-β-D-ribofuranosyl-3-nitropyrrole.

The nucleic acid molecules of the instant invention, individually, or in combination or in conjunction with other drugs, can be used to treat diseases or conditions discussed herein. For example, to treat a particular disease or condition, the siRNA molecules can be administered to a patient or can be administered to other appropriate cells evident to those skilled in the art, individually or in combination with one or more drugs under conditions suitable for the treatment.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition.

By “inhibit”, “down-regulate”, “knockdown” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siRNA) of the invention. In one embodiment, inhibition, down-regulation or reduction with an siRNA molecule is below that level observed in the presence of an inactive or attenuated molecule. In another embodiment, inhibition, down-regulation, or reduction with siRNA molecules is below that level observed in the presence of, for example, an siRNA molecule with scrambled sequence or with mismatches. In another embodiment, inhibition, down-regulation, or reduction of gene expression with a nucleic acid molecule of the instant invention is greater in the presence of the nucleic acid molecule than in its absence.

Gene “silencing” refers to partial or complete loss-of-function through targeted inhibition of gene expression in a cell and may also be referred to as “knockdown”. Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of the invention, including prophylactic and therapeutic methods, which will be capable of knocking down target gene expression, in terms of mRNA levels or protein levels or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siRNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “target nucleic acid” or “nucleic acid target” or “target RNA” or “RNA target” or “target DNA” or “DNA target” is meant any nucleic acid sequence whose expression or activity is to be modulated. The target nucleic acid can be DNA or RNA and is not limited single strand forms.

“Large double-stranded RNA” refers to any double-stranded RNA having a size greater than about 40 bp for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “pyrimidine” as used herein refers to conventional pyrimidines, including uracil and cytosine. In addition, the term pyrimidine is also contemplated to embrace “universal bases” that can be substituted within the compositions and methods of the invention with a pyrimidine. As used herein the term “universal base” refers to nucleotide base analogs that form base pairs with each of the natural DNA/RNA bases with little discrimination between them. A universal base is thus interchangeable with all of the natural bases when substituted into an in an oligonucleotide duplex, typically yielding a duplex which primes DNA synthesis by a polymerase, directs incorporation of the 5′ triphosphate of each of the natural nucleosides opposite the universal base when copied by a polymerase, serves as a substrate for polymerases as the 5′-triphosphate, and is recognized by intracellular enzymes such that DNA containing the universal base can cloned. (Loakes et al., J. Mol Bio 270:426-435 (1997)). In all contexts herein where the term pyrimidine is employed, a universal base may thus be provided as an alternate, chemically modified base target for incorporating into a siRNA of the invention. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, 2001, Nucleic Acids Research, 29:2437-2447).

RNA interference (RNAi) is a biological system that censors the expression of genes by intercepting and destroying the offending messenger RNA (mRNA), without disturbing the mRNA expressed from other genes.

The term “halogen” as used herein refers to bromine, chlorine, fluorine or iodine. In one embodiment, the halogen is chlorine. In another embodiment, the halogen is bromine.

The term “hydroxy” as used herein refers to —OH or —O⁻.

The term “alkyl” as used herein refers to straight- or branched-chain aliphatic groups containing 1-20 carbon atoms, preferably 1-7 carbon atoms and most preferably 1-4 carbon atoms. This definition applies as well to the alkyl portion of alkoxy, alkanoyl and aralkyl groups. In one embodiment, the alkyl is a methyl group.

The term “alkoxy” includes substituted and unsubstituted alkyl, alkenyl, and alkynyl groups covalently linked to an oxygen atom. In one embodiment, the alkoxy group contains 1 to 4 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “nitro”, as used herein alone or in combination refers to a—NO₂ group.

The term “amino” as used herein refers to the group —NRR′, where R and R′ may independently be hydrogen, alkyl, aryl, alkoxy, or heteroaryl. The term “aminoalkyl” as used herein represents a more detailed selection as compared to “amino” and refers to the group —NRR′, where R and R′ may independently be hydrogen or (C₁-C₄)alkyl.

The term “carbonyl” as used herein refers to a group in which an oxygen atom is double-bonded to a carbon atom —O═C.

The term “trifluoromethyl” as used herein refers to —CF₃.

The term “trifluoromethoxy” as used herein refers to —OCF₃.

The term “cycloalkyl” as used herein refers to a saturated cyclic hydrocarbon ring system containing from 3 to 7 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 7 carbon atoms in the cyclic portion and 1 to 4 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(O)-alkyl groups, each optionally containing 2-5 carbon atoms. Specific embodiments of alkanoyl and alkanoyloxy groups are acetyl and acetoxy, respectively.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl; substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present invention include phenyl, substituted phenyl, naphthyl, biphenyl, and diphenyl.

The term “aroyl,” as used alone or in combination herein, refers to an aryl radical derived from an aromatic carboxylic acid, such as optionally substituted benzoic or naphthoic acids.

The term “aralkyl” as used herein refers to an aryl group bonded to the 2-pyridinyl ring and/or the 4-pyridinyl ring through an alkyl group, preferably one containing 1-4 carbon atoms. A preferred aralkyl group is benzyl.

The term “nitrile” or “cyano” as used herein refers to the group —CN.

The term “dialkylamino” refers to an amino group having two attached alkyl groups that can be the same or different.

The term “alkenyl” refers to a straight or branched alkenyl group of 2 to 10 carbon atoms having 1 to 3 double bonds. Preferred embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc.

The term “alkynyl” as used herein refers to a straight or branched alkynyl group of 2 to 10 carbon atoms having 1 to 3 triple bonds. Exemplary alkynyls include, but are not limited to, ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, and 2-decynyl.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄)alkyl.

The term “alkylaminoalkyl” refers to an alkylamino group linked via an alkyl group (i.e., a group having the general structure -alkyl-NH-alkyl or -alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “carboxyalkyl” as used herein refers to the substituent —R′—COOH wherein R′ is alkylene; and carbalkoxyalkyl refers to —R′—COOR wherein R′ and R are alkylene and alkyl respectively. In certain embodiments, alkyl refers to a saturated straight- or branched-chain hydrocarbyl radical of 1-6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxyalkyl” refers to an alkylene group substituted with an alkoxy group. For example, methoxyethyl [CH₃OCH₂CH₂—] and ethoxymethyl (CH₃CH₂OCH₂—] are both C₃ alkoxyalkyl groups.

The term “carboxy”, as used herein, represents a group of the formula —COOH.

The term “alkanoylamino” refers to alkyl, alkenyl or alkynyl groups containing the group —C(O)— followed by —N(H)—, for example acetylamino, propanoylamino and butanoylamino and the like.

The term “carbonylamino” refers to the group —NR—CO—CH₂—R′, where R and R′ may be independently selected from hydrogen or (C₁-C₄)alkyl.

The term “carbamoyl” as used herein refers to —O—C(O)NH₂.

The term “carbamyl” as used herein refers to a functional group in which a nitrogen atom is directly bonded to a carbonyl, i.e., as in —NRC(═O)R′ or —C(═O)NRR′, wherein R and R′ can be hydrogen, alkyl, substituted alkyl, alkenyl, substituted alkenyl, alkoxy, cycloalkyl, aryl, heterocyclo, or heteroaryl.

The term “alkylsulfonylamino” refers to refers to the group —NHS(O)₂Rₐ wherein Rₐ is an alkyl as defined above.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2 or 3 heteroatoms selected from nitrogen atoms, oxygen atoms and sulfur atoms. Plural heteroatoms in a given heterocyclo ring may be the same or different.

Exemplary monocyclic heterocyclo groups include pyrrolidinyl, pyrrolyl, indolyl, pyrazolyl, imidazolyl, oxazolyl, isoxazolyl, thiazolyl, furyl, tetrahydrofuryl, thienyl, piperidinyl, piperazinyl, azepinyl, pyrimidinyl, pyridazinyl, tetrahydropyranyl, morpholinyl, dioxanyl, triazinyl and triazolyl. Preferred bicyclic heterocyclo groups include benzothiazolyl, benzoxazolyl, benzothienyl, quinolinyl, tetrahydroisoquinolinyl, benzimidazolyl, benzofuryl, indazolyl, benzisothiazolyl, isoindolinyl and tetrahydroquinolinyl. In more detailed embodiments heterocyclo groups may include indolyl, imidazolyl, furyl, thienyl, thiazolyl, pyrrolidyl, pyridyl and pyrimidyl.

All value ranges expressed herein, are inclusive over the indicated range. Thus, a range of C₁-C₄ will be understood to include the values of 1, 2, 3, and 4 such that C₁, C₂, C₃ and C₄ are included.

In a further embodiment, the siNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

By “comprising” is meant including, but not limited to, whatever follows the word “comprising.” Thus, use of the term “comprising” indicates that the listed elements are required or mandatory, but that other elements are optional and may or may not be present. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

In this specification and the appended claims, the singular forms of “a”, “an” and “the” include plural reference unless the context clearly dictates otherwise.

### Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small” refers to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology, 211:3-19, Thompson et al., International PCT Publication No. WO 99/54459, Wincott et al., 1995, Nucleic Acids Res., 23:2677-2684, Wincott et al., 1997, Methods Mol. Bio., 74:59, Brennan et al., 1998, Biotechnol Bioeng., 61:33-45, and Brennan, U.S. Pat. No. 6,001,311.

RNA including certain siNA molecules of the invention follows the procedure as described in Usman et al., 1987, J. Am. Chem. Soc., 109:7845; Scaringe et al., 1990, Nucleic Acids Res., 18:5433; and Wincott et al., 1995, Nucleic Acids Res., 23:2677-2684 Wincott et al., 1997, Methods Mol. Bio., 74:59.

Synthesis of universal-binding nucleotide comprising siRNA molecules of the present invention generally follows the procedure described in Usman et al., J. Am. Chem. Soc., 109:7845 (1987); Scaringe et al., Nucleic Acids Res., 18:5433 (1990); and Wincott et al., Nucleic Acids Res., 23:2677-2684 (1995); and Wincott et al., Methods Mol. Bio., 74:59 (1997).

Alternatively, the nucleic acid molecules of the present invention can be synthesized separately and joined together post-synthetically, for example, by ligation (Moore et al., 1992, Science, 256:9923; Draper et al., International PCT Publication No. WO 93/23569; Shabarova et al., 1991, Nucleic Acids Research, 19:4247; Bellon et al., Nucleosides & Nucleotides, 16:951 (1997); Bellon et al., Bioconjugate Chem., 8:204 (1997)), or by hybridization following synthesis and/or deprotection.

Small-Interfering Nucleic Acids (siNAs) and the RISC Complex:

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present invention avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the anti-sense strand of the siRNA duplex. Cleavage of the target RNA takes place in the middle of the region complementary to the anti-sense strand of the siRNA duplex. Studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain non-pairing portions due to mismatch (the corresponding nucleotides are not complementary), bulge (lacking in the corresponding complementary nucleotide on one strand), overhang, and the like. Non-pairing portions can be contained to the extent that they do not interfere with siNA formation. In more detailed embodiments, a “bulge” may comprise 1 to 2 non-pairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other cause are also contemplated. Furthermore, in the present invention the double-stranded region of siNAs in which two strands pair up may contain both bulge and mismatched portions in the approximate numerical ranges specified.

The terminal structure of siNAs of the invention may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (anti-sense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example, Martinez et al., Cell., 110:563-574 (2002) and Schwarz et al., Molecular Cell, 10:537-568 (2002)), or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of the invention lack 2′-hydroxy (2′-OH) containing nucleotides. In certain embodiments short interfering nucleic acids do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within the invention may comprise separate sense and anti-sense sequences or regions, wherein the sense and anti-sense regions are covalently linked by nucleotide or non-nucleotide linker molecules, or are alternately non-covalently linked by ionic interactions, hydrogen bonding, van der waals interactions, hydrophobic interactions, and/or stacking interactions.

siNAs for use within the invention can be assembled from two separate oligonucleotides, where one strand is the sense strand and the other is the anti-sense strand, wherein the anti-sense and sense strands are self-complementary (i.e. each strand comprises nucleotide sequence that is complementary to nucleotide sequence in the other strand; such as where the anti-sense strand and sense strand form a duplex or double stranded structure, for example wherein the double stranded region is about 19 base pairs). The anti-sense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and anti-sense regions of the siNA are linked by means of a nucleic acid-based or non-nucleic acid-based linker(s).

Within additional embodiments, siNAs for intracellular delivery according to the methods and compositions of the invention can be a polynucleotide with a duplex, asymmetric duplex, hairpin or asymmetric hairpin secondary structure, having self-complementary sense and anti-sense regions, wherein the anti-sense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native non-modified siNA, chemically-modified siNA can also minimize the possibility of activating interferon activity in humans.

The siNA molecules described herein, the anti-sense region of a siNA molecule of the invention can comprise a phosphorothioate internucleotide linkage at the 3′-end of said anti-sense region. In any of the embodiments of siNA molecules described herein, the anti-sense region can comprise about one to about five phosphorothioate internucleotide linkages at the 5′-end of said anti-sense region. In any of the embodiments of siNA molecules described herein, the 3′-terminal nucleotide overhangs of a siNA molecule of the invention can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the anti-sense strand, or both strands. The siNA molecules of the invention can comprise one or more phosphorothioate internucleotide linkages at the 3′-end, the 5′-end, or both of the 3′- and 5′-ends of the sense strand, the anti-sense strand, or both strands. For example, an exemplary siNA molecule of the invention can comprise about 1 to about 5 or more (e.g., about 1, 2, 3, 4, 5, or more) consecutive phosphorothioate internucleotide linkages at the 5′-end of the sense strand, the anti-sense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the anti-sense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the anti-sense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the anti-sense strand of the siNA molecules, but also optionally in the sense and/or both anti-sense and sense strands, comprise modified nucleotides having properties or characteristics similar to naturally occurring ribonucleotides. For example, the invention features siNA molecules including modified nucleotides having a Northern conformation (e.g., Northern pseudorotation cycle, see for example Saenger, Principles of Nucleic Acid Structure, Springer-Verlag ed., 1984). As such, chemically modified nucleotides present in the siNA molecules of the invention, preferably in the anti-sense strand of the siNA molecules of the invention, but also optionally in the sense and/or both anti-sense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, ribothymidine nucleotides and 2′-O-methyl nucleotides.

The sense strand of a double stranded siNA molecule may have a terminal cap moiety such as an inverted deoxybasic moiety, at the 3′-end, 5′-end, or both 3′ and 5′-ends of the sense strand.

Non-limiting examples of conjugates include conjugates and ligands described in Vargeese et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the anti-sense strand, or both strands of the chemically-modified siNA molecule. In another embodiment, the conjugate molecule is attached at the 5′-end of either the sense strand, the anti-sense strand, or both strands of the chemically-modified siNA molecule. In yet another embodiment, the conjugate molecule is attached both the 3′-end and 5′-end of either the sense strand, the anti-sense strand, or both strands of the chemically-modified siNA molecule, or any combination thereof. In one embodiment, a conjugate molecule of the invention comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of the invention can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

An siNA further may be further comprised of a nucleotide, non-nucleotide, or mixed nucleotide/non-nucleotide linker that joins the sense region of the siNA to the anti-sense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, for example, Gold et al, Annu. Rev. Biochem., 64:763 (1995); Brody and Gold, J. Biotechnol., 74:5 (2000); Sun, Curr. Opin. Mol. Ther., 2:100 (2000); Kusser, J. Biotechnol., 74:27 (2000); Hermann and Patel, Science, 287:820 (2000); and Jayasena, Clinical Chemistry, 45:1628 (1999)).

A non-nucleotide linker may be comprised of an abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units). Specific examples include those described by Seela and Kaiser, Nucleic Acids Res., 18:6353 (1990) and Nucleic Acids Res., 15:3113 (1987); Cload and Schepartz, J. Am. Chem. Soc., 113:6324 (1991); Richardson and Schepartz, J. Am. Chem. Soc., 113:5109 (1991); Ma et al., Nucleic Acids Res., 21:2585 (1993) and Biochemistry, 32:1751 (1993); Durand et al., Nucleic Acids Res., 18:6353 (1990); McCurdy et al., Nucleosides & Nucleotides, 10:287 (1991); Jschke et al., Tetrahedron Lett., 34:301 (1993); Ono et al., Biochemistry, 30:9914 (1991); Arnold et al., International Publication No. WO 89/02439; Usman et al., International Publication No. WO 95/06731; Dudycz et al., International Publication No. WO 95/11910 and Ferentz and Verdine, J. Am. Chem. Soc., 113:4000 (1991). The synthesis of a siNA molecule of the invention, which can be chemically-modified, comprises: (a) synthesis of two complementary strands of the siNA molecule; and (b) annealing the two complementary strands together under conditions suitable to obtain a double-stranded siNA molecule. In another embodiment, synthesis of the two complementary strands of the siNA molecule is by solid phase oligonucleotide synthesis. In yet another embodiment, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211:3-19, Thompson et al., International PCT Publication No. WO 99/54459, Wincott et al., 1995, Nucleic Acids Res., 23:2677-2684, Wincott et al., 1997, Methods Mol. Bio., 74:59, Brennan et al., 1998, Biotechnol Bioeng., 61:33-45, and Brennan, U.S. Pat. No. 6,001,311. Synthesis of RNA, including certain siNA molecules of the invention, follows general procedures as described, for example, in Usman et al., 1987, J. Am. Chem. Soc., 109:7845; Scaringe et al., 1990, Nucleic Acids Res., 18:5433; and Wincott et al., 1995, Nucleic Acids Res. 23:2677-2684 Wincott et al., 1997, Methods Mol. Bio., 74:59.

The siNAs can be modified extensively to enhance stability by modification with nuclease resistant groups, for example, 2′-amino, 2′-C-allyl, 2′-fluoro, 2′-O-methyl, 2′-H. [For a review see Usman and Cedergren, TIBS, 17:34 (1992); Usman et al., Nucleic Acids Symp. Ser., 31:163 (1994)]. SiNA constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., Eckstein et al., International Publication No. WO 92/07065; Perrault et al., Nature, 344:565 (1990); Pieken et al., Science, 253:314 (1991); Usman and Cedergren, Trends in Biochem. Sci., 17:334 (1992); Usman et al., International Publication No. WO 93/15187; and Rossi et al., International Publication No. WO 91/03162; Sproat, U.S. Pat. No. 5,334,711; Gold et al., U.S. Pat. No. 6,300,074. All of the above references describe various chemical modifications that can be made to the base, phosphate and/or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS, 17:34 (1992); Usman et al., Nucleic Acids Symp. Ser., 31:163 (1994); Burgin et al., Biochemistry, 35:14090 (1996). Sugar modification of nucleic acid molecules have been extensively described in the art (see Eckstein et al., International Publication PCT No. WO 92/07065; Perrault et al., Nature, 344:565-568 (1990); Pieken et al. Science, 253:314-317 (1991); Usman and Cedergren, Trends in Biochem. Sci., 17:334-339 (1992); Usman et al. International Publication PCT No. WO 93/15187; Sproat, U.S. Pat. No. 5,334,711 and Beigelman et al., 1995, J. Biol. Chem., 270:25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., Karpeisky et al., Tetrahedron Lett., 39:1131 (1998); Earnshaw and Gait, Biopolymers (Nucleic Acid Sciences), 48:39-55 (1998); Verma and Eckstein, Annu. Rev. Biochem., 67:99-134 (1998); and Burlina et al., Bioorg. Med. Chem., 5:1999-2010 (1997)). Such publications describe general methods and strategies to determine the location of incorporation of sugar, base and/or phosphate modifications and the like into nucleic acid molecules without modulating catalysis. In view of such teachings, similar modifications can be used as described herein to modify the siNA nucleic acid molecules of the instant invention so long as the ability of siNA to promote RNAi in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, Nucleic Acid Analogues: Synthesis and Properties, in Modern Synthetic Methods, VCH, 331-417 (1995), and Mesmaeker et al., Novel Backbone Replacements for Oligonucleotides, in Carbohydrate Modifications in Anti-sense Research, ACS, 24-39 (1994).

### Administration of Nucleic Acid Molecules

Methods for the delivery of nucleic acid molecules are described in Akhtar et al., Trends Cell Bio., 2:139 (1992); Delivery Strategies for Antisense Oligonucleotide Therapeutics, ed. Akhtar, 1995, Maurer et al., Mol. Membr. Biol., 16:129-140 (1999); Hofland and Huang, Handb. Exp. Pharmacol., 137:165-192 (1999); and Lee et al., ACS Symp. Ser., 752:184-192 (2000), Sullivan et al., PCT WO 94/02595, further describes the general methods for delivery of enzymatic nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as hydrogels, cyclodextrins, biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5:2330-2337 (1999) and Barry et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a patient.

Thus, the invention features a pharmaceutical composition comprising one or more nucleic acid(s) of the invention in an acceptable carrier, such as a stabilizer, buffer, and the like. The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS (Jolliet-Riant and Tillement, Fundam. Clin. Pharmacol., 13:16-26 (1999)); biodegradable polymers, such as poly (DL-lactide-coglycolide) microspheres for sustained release delivery after intracerebral implantation (Emerich, D. F. et al., Cell Transplant, 8:47-58 (1999), Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23:941-949, (1999)). Other non-limiting examples of delivery strategies for the nucleic acid molecules of the instant invention include material described in Boado et al., J. Pharm. Sci., 87:1308-1315 (1998); Tyler et al., FEBS Lett., 421:280-284 (1999); Pardridge et al., PNAS USA., 92:5592-5596 (1995); Boado, Adv. Drug Delivery Rev., 15:73-107 (1995); Aldrian-Herrada et al., Nucleic Acids Res., 26:4910-4916 (1998); and Tyler et al., PNAS USA., 96:7053-7058 (1999).

In accordance with the disclosure herein, the present invention provides novel comprises compositions and methods for inhibiting expression of a target gene in a cell or organism. In related embodiments, the invention provides methods and compositions for treating a subject, including a human cell, tissue or individual, having a disease or at risk of developing a disease caused by the expression of a target gene. In one embodiment, the method includes administering the inventive siRNA or a pharmaceutical composition containing the siRNA to a cell or an organism, such as a mammal, such that expression of the target gene is silenced. Mammalian subjects amendable for treatment using the compositions and methods of the present invention include those suffering from one or more disorders caused by protein overexpression, or which are amenable to treatment by reducing expression of a target protein, including but not limited to, cellular proliferative disorders, differentiative disorders, disorders associated with bone metabolism, immune disorders, hematopoietic disorders, cardiovascular disorders, liver disorders, viral diseases, and metabolic disorders. Exemplary diseases amenable to treatment using modified siRNAs of the invention include various forms of cancer (e.g., carcinomas, sarcomas, metastatic disorders and leukemia). Proliferative disorders amenable to treatment in this context include hematopoietic neoplastic disorders arising from myeloid, lymphoid or erythroid lineages, or precursor cells thereof. Additionally, the methods and compositions of the invention can be employed to treat autoimmune diseases (e.g., diabetes mellitus, rheumatoid arthritis, multiple sclerosis, encephalomyelitis, myasthenia gravis, systemic lupus erythematosis, graft-versus-host disease, and allergies). In other embodiments, the methods and compositions of the invention are effective for treatment and prevention of viral diseases, including but not limited to hepatitis C, hepatitis B, herpes simplex virus (HSV), HIV-AIDS, poliovirus, and smallpox virus. The siRNA compositions and methods of the invention are also useful for treating subjects having an infection or a disease associated with replication or activity of a (+) strand RNA virus having a 3′-UTR, such as HCV. Examples of (+) strand RNA viruses which can be targeted for inhibition include, without limitation, picornaviruses, caliciviruses, nodaviruses, coronaviruses, arteriviruses, flaviviruses, and togaviruses.

Within the methods of the invention for treating and preventing disease, the target gene may be one which is required for initiation or maintenance of the disease, or which has been identified as associated with a higher risk of contracting the disease. In therapeutic methods of the invention, the modified siRNA can be brought into contact directly with the cells or tissues exhibiting the disease. Specific genes which may be targeted for treatment include, but are not limited to oncogenes (Hanahan, D. and R. A. Weinberg, Cell (2000) 100:57; and Yokota, J., Carcinogenesis (2000) 21(3):497-503); cytokine genes (Rubinstein, M., et al., Cytokine Growth Factor Rev. (1998) 9(2):175-81); idiotype (Id) protein genes (Benezra, R., et al., Oncogene (2001) 20(58):8334-41; Norton, J. D., J. Cell Sci. (2000) 113(22):3897-905); prion genes (Prusiner, S. B., et al., Cell (1998) 93(3):337-48; Safar, J., and S. B. Prusiner, Prog. Brain Res. (1998) 117:421-34); genes expressing proteins that induce angiogenesis (Gould, V. E. and B. M. Wagner, Hum. Pathol. (2002) 33(11):1061-3); genes encoding adhesion molecules (Chothia, C. and E. Y. Jones, Annu. Rev. Biochem. (1997) 66:823-62; Parise, L. V., et al., Semin. Cancer Biol. (2000) 10(6):407-14); genes encoding cell surface receptors (Deller, M. C., and Y. E. Jones, Curr. Opin. Struct. Biol. (2000) 10(2):213-9); genes encoding proteins involved in metastatic and/or invasive processes (Boyd, D., Cancer Metastasis Rev. (1996) 15(1):77-89; Yokota, J., Carcinogenesis (2000) 21(3):497-503); genes encoding proteases, molecules that regulate apoptosis, or proteins that regulate the cell cycle (Matrisian, L. M., Curr. Biol. (1999) 9(20): R.sub.776-8; Krepela, E., Neoplasma (2001) 48(5):332-49; Basbaum and Werb, Curr. Opin. Cell Biol. (1996) 8:731-738; Birkedal-Hansen, et al., Crit. Rev. Oral Biol. Med. (1993) 4:197-250; Mignatti and Rifkin, Physiol. Rev. (1993) 73:161-195; Stetler-Stevenson, et al., Annu. Rev. Cell Biol. (1993) 9:541-573; Brinkerhoff, E., and L. M. Matrisan, Nature Reviews (2002) 3:207-214; Strasser, A., et al., Annu. Rev. Biochem. (2000) 69:217-45; Chao, D. T. and S. J. Korsmeyer, Annu. Rev. Immunol. (1998) 16:395-419; Mullauer, L., et al., Mutat. Res. (2001) 488(3):211-31; Fotedar, R., et al., Prog. Cell Cycle Res. (1996) 2:147-63; Reed, J. C., Am. J. Pathol. (2000) 157(5):1415-30; D'Ari, R., Bioassays (2001) 23(7):563-5); genes encoding EGF receptors (Mendelsohn, J. and J. Baselga, Oncogene (2000) 19(56):6550-65; Normanno, N., et al., Front. Biosci. (2001) 6:D685-707); and multi-drug resistance genes (Childs, S., and V. Ling, Imp. Adv. Oncol. (1994) 21-36), among other disease-associated gene targets.

Typically, the dose range of the siRNA will be in the range of 0.001 to 500 milligrams per kilogram/day (e.g., about 1 microgram per kilogram to about 500 milligrams per kilogram, about 100 micrograms per kilogram to about 100 milligrams per kilogram, about 1 milligram per kilogram to about 75 milligrams per kilogram, about 10 micrograms per kilogram to about 50 milligrams per kilogram, or about 1 microgram per kilogram to about 50 micrograms per kilogram). Dosage levels of the order of from about 0.1 mg to about 140 mg per kilogram of body weight per day are useful in the treatment of the above-indicated conditions (about 0.5 mg to about 7 g per patient per day). These and other effective unit dosage amounts may be administered in a single dose, or in the form of multiple daily, weekly or monthly doses, for example in a dosing regimen comprising from 1 to 5, or 2-3, doses administered per day, per week, or per month. The dosing schedule may vary depending on a number of clinical factors, such as the subject's sensitivity to the protein. Examples of dosing schedules are 3 μg/kg administered twice a week, three times a week or daily; a dose of 7 μg/kg twice a week, three times a week or daily; a dose of 10 μg/kg twice a week, three times a week or daily; or a dose of 30 μg/kg twice a week, three times a week or daily. Following administration of the siRNA composition according to the formulations and methods of the invention, test subjects will exhibit a 10%, 20%, 30%, 50% or greater reduction, up to a 75-90%, or 95% or greater, reduction, in one or more symptoms associated with the disease, as compared to placebo-treated or other suitable control subjects.

Within additional aspects of the invention, combinatorial formulations and coordinate administration methods are provided which employ an effective amount of siRNA, and one or more additional active agent(s) that is/are combinatorially formulated or coordinately administered with the siRNA—yielding an effective formulation or method to modulate, alleviate, treat or prevent the disease in a mammalian subject. Exemplary combinatorial formulations and coordinate treatment methods in this context employ the siRNA in combination with one or more additional or adjunctive therapeutic agents. The secondary or adjunctive methods and compositions useful in the treatment of diseases caused by the overexpression of proteins include, but are not limited to, combinatorial administration with enzymatic nucleic acid molecules, allosteric nucleic acid molecules, anti-sense, decoy, or aptamer nucleic acid molecules, antibodies such as monoclonal antibodies, small molecules, and other organic and/or inorganic compounds including metals, salts and ions.

To practice the coordinate administration methods of the invention, a siRNA is administered, simultaneously or sequentially, in a coordinate treatment protocol with one or more of the secondary or adjunctive therapeutic agents contemplated herein. The coordinate administration may be done in either order, and there may be a time period while only one or both (or all) active therapeutic agents, individually and/or collectively, exert their biological activities. A distinguishing aspect of all such coordinate treatment methods is that the siRNA present in the composition elicits some favorable clinical response, which may or may not be in conjunction with a secondary clinical response provided by the secondary therapeutic agent. Often, the coordinate administration of the siRNA with a secondary therapeutic agent as contemplated herein will yield an enhanced therapeutic response beyond the therapeutic response elicited by either or both the purified siRNA and/or secondary therapeutic agent alone.

In addition to in vivo gene inhibition, the skilled artisan will appreciate that the modified siRNA agents of the present invention are useful in a wide variety of in vitro applications. Such in vitro applications, include, for example, scientific and commercial research (e.g., elucidation of physiological pathways, drug discovery and development), and medical and veterinary diagnostics. In general, the method involves the introduction of the siRNA agent into a cell using known techniques (e.g., absorption through cellular processes, or by auxiliary agents or devices, such as electroporation, lipofection, or through the use of peptide conjugates), then maintaining the cell for a time sufficient to obtain degradation of an mRNA transcript of the target gene.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev., 95:2601-2627 (1995); Ishiwata et al., Chem. Pharm. Bull., 43:1005-1011 (1995)). Such liposomes have been shown to accumulate selectively in tumors, presumably by extravasation and capture in the neovascularized target tissues (Lasic et al., Science, 267:1275-1276 (1995); Oku et al., Biochim. Biophys. Acta, 1238:86-90 (1995)). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42:24864-24870 (1995); Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985). For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between 0.1 mg/kg and 100 mg/kg body weight/day of active ingredients is administered dependent upon potency of the negatively charged polymer.

The present invention also includes compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985), hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize.

The universal-binding nucleotide comprising siRNA molecules of the instant invention can be used as pharmaceutical agents. Pharmaceutical agents prevent, modulate the occurrence, or treat (alleviate a symptom to some extent, preferably all of the symptoms) of a disease state in a patient.

The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

Other non-limiting examples of delivery strategies for the nucleic acid molecules of the instant invention include material described in Boado et al., J. Pharm. Sci., 87:1308-1315 (1998); Tyler et al., FEBS Lett., 421:280-284 (1999); Pardridge et al., PNAS USA, 92:5592-5596 (1995); Boado, Adv. Drug Delivery Rev., 15:73-107 (1995); Aldrian-Herrada et al., Nucleic Acids Res., 26:4910-4916 (1998); and Tyler et al., PNAS USA., 96:7053-7058 (1999).

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

In one embodiment, the invention compositions suitable for administering nucleic acid molecules of the invention to specific cell types, such as hepatocytes. For example, the asialoglycoprotein receptor (ASGPr) (Wu and Wu, J. Biol. Chem. 262:4429-4432 (1987)) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, Cell, 22:611-620 (1980); Connolly et al., J. Biol. Chem., 257:939-945 (1982)). Lee and Lee, Glycoconjugate J., 4:317-328 (1987), obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., J. Med. Chem., 24:1388-1395 (1981)). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavailability, pharmacodynamics, and pharmacokinetic parameters can be modulated through the use of nucleic acid bioconjugates of the invention.

### Supplemental or Complementary Methods of Delivery

Supplemental or complementary methods for delivery of nucleic acid molecules for use within then invention are described, for example, in Akhtar et al., Trends Cell Bio., 2:139 (1992); Delivery Strategies for Anti-sense Oligonucleotide Therapeutics, ed. Akhtar, 1995, Maurer et al., Mol. Membr. Biol., 16:129-140 (1999); Hofland and Huang, Handb. Exp. Pharmacol., 137:165-192 (1999); and Lee et al., ACS Symp. Ser., 752:184-192 (2000). Sullivan et al., International PCT Publication No WO 94/02595, further describes general methods for delivery of enzymatic nucleic acid molecules. These protocols can be utilized to supplement or complement delivery of virtually any nucleic acid molecule contemplated within the invention.

Nucleic acid molecules and polypeptides can be administered to cells by a variety of methods known to those of skill in the art, including, but not restricted to, administration within formulations that comprise the siNA and polypeptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5:2330-2337 (1999) and Barry et al., International PCT Publication No. WO 99/31262.

The compositions of the instant invention can be effectively employed as pharmaceutical agents. Pharmaceutical agents prevent, modulate the occurrence or severity of, or treat (alleviate one or more symptom(s) to a detectable or measurable extent) of a disease state or other adverse condition in a patient.

Thus within additional embodiments the invention provides pharmaceutical compositions and methods featuring the presence or administration of one or more polynucleic acid(s), typically one or more siNAs, combined, complexed, or conjugated with a polypeptide, optionally formulated with a pharmaceutically-acceptable carrier, such as a diluent, stabilizer, buffer, and the like.

The present invention satisfies additional objects and advantages by providing short interfering nucleic acid (siNA) molecules that modulate expression of genes associated with a particular disease state or other adverse condition in a subject. Typically, the siNA will target a gene that is expressed at an elevated level as a causal or contributing factor associated with the subject disease state or adverse condition. In this context, the siNA will effectively downregulate expression of the gene to levels that prevent, alleviate, or reduce the severity or recurrence of one or more associated disease symptoms. Alternatively, for various distinct disease models where expression of the target gene is not necessarily elevated as a consequence or sequel of disease or other adverse condition, down regulation of the target gene will nonetheless result in a therapeutic result by lowering gene expression (i.e., to reduce levels of a selected mRNA and/or protein product of the target gene). Alternatively, siNAs of the invention may be targeted to lower expression of one gene, which can result in upregulation of a “downstream” gene whose expression is negatively regulated by a product or activity of the target gene.

Thus siNAs of the present invention may be administered in any form, for example transdermally or by local injection. Comparable methods and compositions are provided that target expression of one or more different genes associated with a selected disease condition in animal subjects, including any of a large number of genes whose expression is known to be aberrantly increased as a causal or contributing factor associated with the selected disease condition.

The siNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

In more detailed aspects of the invention, the mixture, complex or conjugate comprising a siRNA and a polypeptide can be optionally combined with (e.g., admixed or complexed with) a cationic lipid, such as LIPOFECTIN®. To produce these compositions comprised of a polypeptide, siRNA and a cationic lipid, the siRNA and peptide may be mixed together first in a suitable medium such as a cell culture medium, after which the cationic lipid is added to the mixture to form a siRNA/delivery peptide/cationic lipid composition. Optionally, the peptide and cationic lipid can be mixed together first in a suitable medium such as a cell culture medium, whereafter the siRNA can be added to form the siRNA/delivery peptide/cationic lipid composition.

In another embodiment, a small nucleic acid molecule, such as short interfering nucleic acid (siNA), a short interfering RNA (siRNA), a double-stranded RNA (dsRNA), micro-RNA (mRNA), or a short hairpin RNA (shRNA), admixed or complexed with the polypeptide and one or more non-cationic lipids or a combination of a non-cationic lipid and a cationic lipid to form a composition that enhances intracellular delivery of the siNA as compared to delivery resulting from contacting the target cells with a naked siNA (i.e., siNA without a polypeptide and/or a non-cationic lipid present). The siNA may also be conjugated to the polypeptide and admixed with one or more non-cationic lipids or a combination of a non-cationic lipid and a cationic lipid to form a composition that enhances intracellular delivery of the siNA as compared to delivery resulting from contacting the target cells with a naked siNA.

These and other subjects are effectively treated, prophylactically and/or therapeutically, by administering to the subject an effective amount of one or more siRNA(s) of the invention containing a multiply-modified ribonucleotide according to Formula I. Within additional aspects of the invention, combinatorial formulations and methods are provided comprising an effective amount of one or more siRNA(s) of the present invention in combination with one or more secondary or adjunctive active agent(s) that is/are combinatorially formulated or coordinately administered with the siRNA(s) to control a targeted disease or condition as described herein. Useful adjunctive therapeutic agents in these combinatorial formulations and coordinate treatment methods include, for example, enzymatic nucleic acid molecules, allosteric nucleic acid molecules, anti-sense, decoy, or aptamer nucleic acid molecules, antibodies such as monoclonal antibodies, small molecules, and other organic and/or inorganic compounds including metals, salts and ions, and other drugs and active agents indicated for treating a targeted disease or condition, including but not limited to, carboplatin, cisplatin, etoposide, gemcitabine, irinotecan, taxol, paclitaxel, docetaxel, vinorelbine, and gefitinib.

In another embodiment, an siRNA of the invention includes a conjugate member on one or more of the terminal nucleotides of the siRNA. The conjugate member can be, for example, a lipophile, a terpene, a protein binding agent, a vitamin, a carbohydrate, or a peptide. For example, the conjugate member can be naproxen, nitroindole (or another conjugate that contributes to stacking interactions), folate, ibuprofen, or a C5 pyrimidine linker. In other embodiments, the conjugate member is a glyceride lipid conjugate (e.g., a dialkyl glyceride derivatives), vitamin E conjugates, or thio-cholesterols. Additional conjugate members include peptides that function, when conjugated to a modified siRNA of the invention, to facilitates delivery of the siRNA into a target cell, or otherwise enhance delivery, stability, or activity of the siRNA when contacted with a biological sample (e.g., a target cell). Exemplary peptide conjugate members for use within these aspects of the invention, including, but not limited to peptides PN27, PN28, PN29, PN58, PN61, PN73, PN158, PN159, PN173, PN182, PN202, PN204, PN250, PN361, PN365, PN404, PN453, and PN509, are described, for example, in U.S. patent application Ser. No. 11/121,566 filed May 4, 2005, and U.S. application Ser. No. 11/107,371 filed Apr. 15, 2005 and are incorporated herein by reference. When peptide conjugate partners are used to enhance delivery of modified siRNAs of the invention, the resulting siRNA formulations and methods will often exhibit further reduction of an interferon response in target cells as compared to siRNAs delivered in combination with alternate delivery vehicles, such as lipid delivery vehicles (e.g., lipofectamine).

Compositions Comprising Universal-Binding Nucleotide Comprising siRNA

Universal-binding nucleotide comprising siRNA of the present invention, either individually or in combination with one or more other compound, can be used to treat diseases or conditions as discussed herein or as otherwise known in the art. To treat a particular disease or condition, the universal-binding nucleotide comprising siRNA molecules can be administered to a patient or can be administered to other appropriate cells evident to those skilled in the art, individually or in combination with one or more compound under conditions suitable for the treatment.

For example, the universal-binding nucleotide comprising siRNA molecules described herein can be used in combination with other known treatments and/or therapeutic agents to treat a wide variety of conditions, particularly viral infections. Non-limiting examples of other therapeutic agents that can be readily combined with a universal-binding nucleotide comprising siRNA molecule of the invention include, for example, enzymatic nucleic acid molecules; allosteric nucleic acid molecules; anti-sense, decoy, or aptamer nucleic acid molecules; antibodies such as monoclonal antibodies; small molecules; and other organic and/or inorganic compounds including metals, salts and ions.

Thus, the invention features compositions comprising one or more universal-binding nucleotide comprising siRNA molecules of the invention in an acceptable carrier, such as a stabilizer, buffer, and the like. The negatively charged siRNA molecules of the invention may be administered to a patient by any standard means, with or without stabilizers, buffers, and the like, to form a composition suitable for treatment. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, and suspensions for injectable administration, either with or without other compounds known in the art.

The present invention also includes pharmaceutically acceptable formulations of the compounds and compositions described herein. These formulations include salts of the above compounds, e.g., acid addition salts such as salts of hydrochloric acid, hydrobromic acid, acetic acid, and benzene sulfonic acid.

A pharmaceutical composition or formulation refers to a composition or formulation in a form suitable for administration, e.g., systemic administration, into a cell or patient such as a human. Suitable forms, in part, depend upon the use or the route of entry, for example oral, transdermal, or by injection. Such forms should not prevent the composition or formulation from reaching a target cell (i.e, a cell to which the negatively charged nucleic acid is desirable for delivery). For example, pharmaceutical compositions injected into the blood stream should be soluble. Other factors are known in the art, and include considerations such as toxicity and forms that prevent the composition or formulation from exerting its effect.

Methods for Selecting Universal-Binding Nucleotide Comprising siRNA

As indicated above, the present invention also provides methods for selecting modified siRNA molecules that are capable of specifically binding to a wide range of desired gene target variants while being incapable of specifically binding to non-desired gene target variants. The selection process disclosed herein is useful, for example, in eliminating modified siRNAs that are capable of exerting a cytotoxic effect resulting from non-specific binding to, and subsequent degradation of, one or more non-target gene.

Certain embodiments disclosed herein provide methods for selecting one or more modified siRNA molecule(s) that employ the step of predicting the stability of an siRNA duplex. Typically, such a prediction is achieved by employing a theoretical melting curve wherein a higher theoretical melting curve indicates an increase in siRNA duplex stability and a concomitant decrease in cytotoxic effects. Alternatively, stability of an siRNA duplex may be determined empirically by measuring the hybridization of a single modified RNA strand containing one or more universal-binding nucleotide(s) to a complementary target gene within, for example, a polynucleotide array. The melting temperature (i.e., the Tₘ value) for each modified RNA and complementary RNA immobilized on the array can be determined and, from this Tₘ value, the relative stability of the modified RNA pairing with a complementary RNA molecule determined.

Kawase et. al. have described an analysis of the nucleotide-pairing properties of dl to A, C, G, and T, which was achieved by measuring the hybridization of oligonucleotides (ODNs) with dI in various positions to complementary sets of ODNs made as an array. Nucleic Acids Research, 14:7727-7736 (1986). The relative strength of nucleotide-pairing is I-C>I-A>I-G˜I-T. Generally, dI containing duplexes showed lower Tm values when compared to the corresponding WC nucleotide pair. The stabilization of dI by pairing was in order of dC>dA>dG>dT>dU. (See, Table 1).

TABLE 1
d (GGAAAAXAAAAGG)
d (CCTTTTYTTTTCC)

| | | Corresponding | | Corresponding | |
|---|---|---|---|---|---|
| Duplex X/Y | | WT sequence | | WT sequence | |
| nucleotide | Tₘ° | where | Tₘ° | where | Tₘ° |
| pair | C. | X/Y are | C. | X/Y are | C. |
| I/C | 50.9 | G/C | 52.8 | | |
| I/A | 47.0 | T/A | 52.8 | U/A | 51.0 |
| I/G | 43.8 | G/C | 52.8 | | |
| I/T | 43.4 | A/T | 52.8 | A/U | 51.0 |
| I/U | 39.7 | A/U | 51.0 | | |

The following rules, derived from Kawase et al., are applicable to the design and selection of universal-binding nucleotide comprising siRNA according to the present invention, wherein the universal-binding nucleotide is insosine: (a) when XY=IC, Tₘ (A₂₆₀=0.5) is measured to be 51.1° C. while the corresponding wild type double-strand siRNA melts at 59.2° C., an approximately 4° decrease per substitution in the melting temperature; (b) when XY=IA, Tₘ (A₂₆₀=0.5) is measured to be 44.7° C. while the corresponding wild type double-strand siRNA melts at 42.3° C. (that is, replacement of two Ts with dI in the self-complementary duplex shown in Table 2 stabilizes the duplex marginally—˜1.2° C. per substitution); (c) when XY=IG, Tₘ (A₂₆₀=0.5) is measured to be only 35.0° C. while the corresponding wild type double-strand siRNA (XY=CG) melts at 51.0° C., an approximately 8° C. decrease per substitution in the melting temperature; (d) when XY=IT, the siRNA duplex is not expected to show cooperative melting, but the wild sequence (XY=AT) melts at 54.8° C. (indicating that the I-T nucleotide pair is very unstable—that is, replacement of 2 As in the siRNA duplex with two dIs; (e) incorporation of 4 dI in the duplex presented in Table 2 destabilizes the duplex significantly.

From the thermodynamic values calculated using van't Hoff plots according to a two state model, Kawase et al. conclude that the sequence of purine-pyrimidine is favored in double strand formation due to nucleotide stacking. For instance the duplex formation of XY=AT is more favored than XY=CG and TA. (See, Table 2)

TABLE 2
Tₘ values of self-complementary duplexes

| | Tₘ | Tₘ | Tₘ | Tₘ | Tₘ |
|---|---|---|---|---|---|
| | (A₂₆₀ = | (A₂₆₀ = | (A₂₆₀ = | (A₂₆₀ = | (A₂₆₀ = |
| d(GGGAAXYTTCCC) | 0.25) | 0.5) | 1.0) | 2.0) | 3.0) |
| IC | 48.5 | 51.1 | 52.6 | 55.0 | 55.8 |
| IA | 42.5 | 44.7 | 45.8 | 48 | 49.0 |
| IG | — | 35.0 | 36.5 | 38.3 | 39.7 |
| IT | — | — | — | — | — |
| II | — | — | — | — | — |
| GC | 56.5 | 59.2 | 60.7 | 62.8 | 63.5 |
| GA | 42.0 | 44.1 | 45.9 | 48.5 | 50.3 |
| GG | — | 33.2 | 36.7 | 38.4 | 40.8 |
| GT | — | — | — | — | — |
| AT | 51.6 | 54.8 | 57.0 | 58.0 | 58.8 |
| TA | 40.6 | 42.3 | 43.9 | 45.2 | 45.9 |
| CG | 50.4 | 51.0 | 52.2 | 55.5 | 56.2 |
| AC | — | — | — | — | — |
| CT | — | — | — | — | — |

Note 1:
Tₘs were measured at various concentrations and have been shown by their A₂₆₀.
Note 2:
Where there is no date, the duplex did not show cooperative melting.

Alternative embodiments provide methods for selecting one or more universal-binding nucleotide comprising siRNA, which methods employ “off-target” profiling whereby one or more universal-binding nucleotide comprising siRNA is administered to a cell(s), either in vivo or in vitro, and total mRNA is collected, and used to probe a microarray comprising oligonucleotides having one or more nucleotide sequence from a panel of known genes, including non-target genes. The “off-target” profile of the modified siRNA is quantified by determining the number of non-target genes having reduced expression levels in the presence of the universal-binding nucleotide comprising siRNA. The existence of “off target” binding indicated an siRNA that is capable of specifically binding to one or more non-target gene. Ideally, a universal-binding nucleotide comprising siRNA applicable to therapeutic use will exhibit a high Tₘ value while exhibiting little or no “off-target” binding.

Still further embodiments provide methods for selecting one or more potentially efficacious universal-binding nucleotide comprising siRNA. Such methods employ one or more reporter gene construct comprising a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide (typically between about 15 base-pairs and about 40 base-pairs, more typically between about 19 base-pairs and about 30 base-pairs, most typically 20, 21, 22, 23, 24, 25, 26, 27, 28, or 29 base-pairs) that contains a target sequence for the one or more universal-binding nucleotide comprising siRNA.

Individual reporter gene expression constructs may be co-transfected with one or more universal-binding nucleotide comprising siRNA. The capacity of a given universal-binding nucleotide comprising siRNA to reduce the expression level of each of the contemplated gene variants may be determined by comparing the measured reporter gene activity from cells transfected with and without the modified siRNA.

Within other aspects of the present invention are provided methods that employ one or more siRNA, and compositions comprising one or more siRNA, wherein at least one of the siRNA comprise one or more universal-binding nucleotide(s) in the first, second and/or third position in the anti-codon of the anti-sense strand of the siRNA duplex is capable of specifically binding to an mRNA, such as an mRNA expressed by a target virus.

Within certain embodiments, methods disclosed herein comprise the steps of (a) selecting a target gene, wherein the target gene is a target viral gene, for siRNA-mediated gene silencing; (b) designing and/or synthesizing a suitable siRNA for siRNA gene silencing of the target viral gene, wherein the siRNA comprises one or more universal-binding nucleotide in the anti-sense strand; and (c) administering the siRNA to a cell expressing the target virus gene, wherein the siRNA is capable of specifically binding to the target virus gene thereby reducing its expression level in the cell.

Within alternative embodiments, methods disclosed herein comprise the steps of (a) selecting a target gene for siRNA-mediated gene silencing, wherein the target gene is an endogenous gene wherein the endogenous target gene comprises one or more sequence variation from a corresponding wild-type endogenous gene; (b) designing and/or synthesizing a suitable siRNA for siRNA gene silencing of the endogenous target gene, wherein the siRNA comprises one or more universal-binding nucleotide in the anti-sense strand; and (c) administering the siRNA to a cell expressing the endogenous target gene, wherein the siRNA is capable of specifically binding to the endogenous target gene thereby reducing its expression level in the cell.

It will be understood that methods of the present invention do not require a priori knowledge of the nucleotide sequence of every possible gene variant(s) targeted by the universal-binding nucleotide comprising siRNA. Initially, the nucleotide sequence of the siRNA is selected from a conserved region of the target gene.

Within certain embodiments of the presently disclosed methods, one or more anti-codon(s) within the anti-sense strand of the siRNA molecule is modified by substituting a universal-binding nucleotide for a first position (i.e., the wobble nucleotide position) in the anti-codon(s) of the anti-sense strand. Relying on the wobble hypothesis, the first nucleotide-pair substitution allows the “modified siRNA” anti-sense strand to specifically bind to RNA wherein a first nucleotide-pair substitution has occurred, but which substitution does not result in an amino acid change in the corresponding gene product owing to the redundancy of the genetic code.

Within alternative embodiments of the presently disclosed methods, one or more anti-codon(s) within the anti-sense strand of the siRNA molecule is modified by substituting a universal-binding nucleotide for a second and/or third position in the anti-codon(s) of the anti-sense strand. By substituting a universal-binding nucleotide for a first and/or second position, the one or more first and/or second position nucleotide-pair substitution allows the “modified siRNA” molecule to specifically bind to mRNA wherein a first and/or a second position nucleotide-pair substitution has occurred, wherein the one or more nucleotide-pair substitution results in an amino acid change in the corresponding gene product.

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### EXAMPLE 1

### Preparation of Melamine Derivatives

### Methods and Materials for 2,4,6-Triamidosarcocyl Melamine

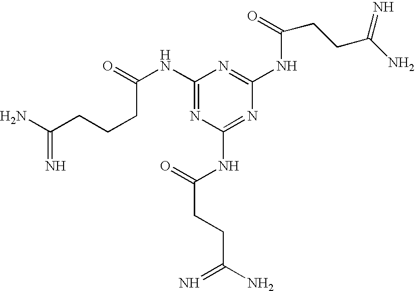

4-Methoxy-2,3,6-trimethylbenzenesulfonyl (Mtr) Creatine

A solution of creatine (390 mgs-3 mmol) in a mixture of 4N NaOH (3 ml) and acetone is cooled in an ice water bath and treated with Mtr chloride (680 mgs-5.25 mmol) in acetone (3 mls). The mixture is stirred overnight at room temperature and then acidified with 10% citric acid in water. The acetone is evaporated and the residual aqueous suspension is extracted with ethyl acetate, 3×10 ml. The combined extracts are dried over magnesium sulfate, filtered and the filtrate is evaporated to dryness. The residue is crystallized from ethyl acetate:hexane.

### 2,4,6-Mtr-triamidosarcocyl Melamine

The Mtr-creatine (694 mgs-2 mmol) is dissolved in 5 ml of dimethylformamide (DMF) with melamine (76 mgs-0.6 mmol), hydroxybenzotriazole (310 mgs-2 mmol) and diisopropylethylamine (403 ul-2.3 mmol). With the addition of diisopropylcarbodiimide (DIC) (310 ul-2 mmol) the mixture is stirred overnight at room temperature.

The next day the reaction is diluted with 50 ml of ethyl acetate, extracted 3×10 ml of 10% citric acid, 1× brine, 3×10% sodium bicarbonate and 1× brine. The ethyl acetate is dried over magnesium sulfate, filtered, evaporated and the residue is crystallized from ether:hexane.

### 2,4,6-Triamidosarcocyl Melamine

The 2,4,6-Mtr-triamidosarcocyl melamine (340 mgs-0.3 mmol) is dissolved in trifluoroacetic acid:thianisole (95:5) (5 ml) and stirred of for four hours. The solution is evaporated to an oil and triturated with ether and dried.

### Methods and Materials for 2,4,6-Triguanidino Triazine

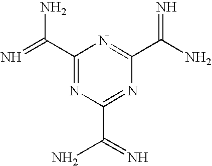

### Melamine Trithiourea Sulfonic Acid

A mixture of melamine (1620 mgs-13 mmol) is and methyl thiocynate (2870 mgs-139 mmol) in 70 mls of ethyl alcohol is refluxed for one hour. After evaporation the corresponding urea is isolated by evaporation of the alcohol. The triisothiourea triazine intermediate is then dissolved in water (10 ml) containing sodium chloride (mg-mmol), sodium molybdate dehydrate and cooled to 0° C. with vigorous stirring. Hydrogen peroxide (30%-41 mmol) is added dropwise to the stirring suspension. The sulfonic acid product is collected by filtration and washed with cold brine and dried.

### 2,4,6-Triguanidino Triazine

The melamine trithiourea sulfonic acid (1520 mgs-10 mmol) is added to the appropriate amine (13 mmol) in 5 ml of acetonitrile at room temperature. The mixture is stirred overnight. The pH is adjusted to 12 with 3N NaOH. Depending on the amine used, the guanidine product can be filtered of a s solid or extracted with methylene chloride for isolation purposes.

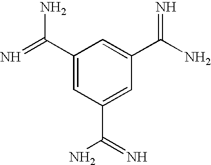

### EXAMPLE 2

### Effective In Vitro Knockdown of β-Galactosidase Activity by a Modified LacZ siRNA

Beta-Gal siRNA Sequence

The double-stranded siRNA sequences shown below were produced synthesize using standard techniques. The siRNA sequences were designed to silence the beta galactosidase mRNA. The siRNAs were encapsulated in lipofectamine to promote transfection of the siRNA into the cells. The sequences are identical except for the varied substitution of ribose uracils by ribose thymines. The siRNA of duplex 4 did not replace any of the ribose uracils with ribose thymine. The siRNAs of duplexes 1-3 represent siRNAs of the present invention in which some or all of the uracils present in duplex 4 have been changed to ribose thymines. All of the uracils have been changed to ribose thymines in the siRNA of duplex 1. Only the uracils in the sense strand have been changed to ribose thymines in the siRNA of duplex 2. In duplex 3 only the uracils in the antisense strand were changed to ribose thymines. The purpose of the present experiment was to determine which siRNAs would be effective in silencing the β-galactosidase mRNA.

1. Duplex 1

(SEQ ID NO:1)

C.rT.A.C.A.C.A.A.A.rT.C.A.G.C.G.A.rT.rT.rT.dT.dT

(SEQ ID NO:2)

A.A.A.rT.C.G.C.rT.G.A.rT.rT.rT.G.rT.G.rT.A.G.dT.dT
2. Duplex 2

(SEQ ID NO:3)

C.rT.A.C.A.C.A.A.A.rT.C.A.G.C.G.A.rT.rT.rT.dT.dT

(SEQ ID NO:4)

A.A.A.U.C.G.C.U.G.A.U.U.U.G.U.G.U.A.G.dT.dT
3. Duplex 3

(SEQ ID NO:5)

C.U.A.C.A.C.A.A.A.U.C.A.G.C.G.A.U.U.U.dT.dT

(SEQ ID NO:6)

A.A.A.rT.C.G.C.rT.G.A.rT.rT.rT.G.rT.G.rT.A.G.dT.dT
4. Duplex 4

(SEQ ID NO:7)

C.U.A.C.A.C.A.A.A.U.C.A.G.C.G.A.U.U.U.dT.dT

(SEQ ID NO:8)

A.A.A.U.C.G.C.U.G.A.U.U.U.G.U.G.U.A.G.dT.dT

### Procedure

β-Gal Activity Assay Protocol for 9LacZR Cells

9lacZ/R cells were seeded in 6-well collagen-coated plates with 5×10e⁵ cells/well (2 mls total per well) and cultured with DMEM/high glucose media at 37° C. and 5% CO₂ overnight.

Preparation for transfection: 250 μl of Opti-MEM media without serum was mixed with 5 μl of 20 pmol/μl siRNA and 5 μl of Lipofectamine is mixed with another 250 μl Opti-MEM media. After both mixtures were allowed to equilibrate for 5 min, tubes were then mixed and left at room temperature for 20 min to form transfection complexes. During this time, complete media was aspirated from 6 well plates and cells were washed with incomplete Opti-MEM. 500 μl of transfection mixture were applied to wells and cells were left at 37° C. for 4 hrs. To ensure adequate coverage cells were gently shaken or rocked during this incubation.

After 4 hr incubation, the transfection media was washed once with complete DMEM/high glucose media and then replaced with the same media. The cells were then incubated for 48 hrs at 37°, 5% CO2.

### β-Galactosidase Assay (Invitrogene Assay Kit)

Transfected cells were washed with PBS, and then harvested with 0.5 mls of trypsin/EDTA. Once the cells were detached, 1 ml of complete DMEM/high glucose was added per well and the samples were transferred to microfuge tubes. The samples were then spun at 250×g for 5 minutes and the supernatant was then removed. The cells were resuspended in 50 μl of 1× lysis buffer at 4° C. The samples were then freeze-thawed with dry ice and a 37° water bath 2 times. After freeze-thawing, the samples were centrifuged for 5 minutes at 4° C. and the supernatant was transferred to a new microcentrifuge tube.

For each sample, 1.5 and 10 μl of lysate were transferred to a fresh tube and made up each sample to a final volume of 30 μl with sterile water. Add 70 μl of ONPG and 200 μl of 1× cleavage buffer with β-mercaptoethanol and mixed briefly, then incubated samples for 30 min. at 37° C. After incubation, add 500 μl of stop buffer for a final of 800 μl. Samples were then read in disposable cuvettes at 420 nm.

For the purpose of the instant example, the level of measured LacZ activity was correlated with the quantity of LacZ transcript within 9L/LacZ cells. Thus, a reduction in LacZ activity after siRNA transfection, without having a negative impact on cell viability, was attributed to a reduction in the quantity of LacZ transcripts via targeted degradation mediated by the LacZ siRNA.

### Protein

Protein concentration was determined by BCA method.

### Results

All of the siRNA were effective in silencing the β-galactosidase mRNA.

### EXAMPLE 3

### Stability of siRNA in Rat Plasma

### Purpose

The purpose of this experiment was to determine how stable the siRNAs of Example 2 were to the ribonucleases present in rat plasma.

A 20 μg aliquot of each siRNA duplex of example 2 was mixed with 200 μl of fresh rat plasma incubated at 37° C. At various time points (0, 30, 60 and 20 min), 50 μl of the mixture was taken out and immediately extracted by phenol:chloroform. SiRNAs were dried following precipitation by adding 2.5 volume of isopropanol alcohol and subsequent washing step with 70% ethanol. After dissolving in water and gel loading buffer the samples were analyzed on 20% polyacrylamide gel, containing 7 M urea and visualized by ethidium bromide staining and quantitated by densitometry.

### Results

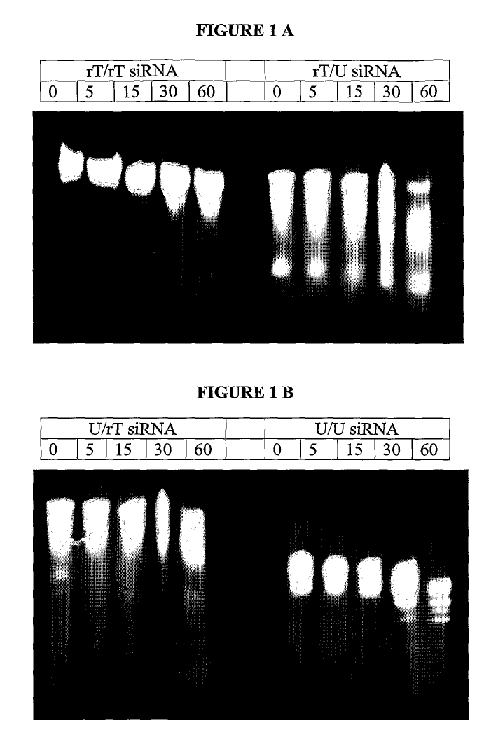

FIG. 1 shows the level of degradation at each time point for each of the constructs on a PAGE gel. Both the double strand modified (rT/rT; A) and single strand modified (U/rT and rT/U, A and B) siRNAs show little to no degradation after treatment with plasma. In contrast, the non-modified (siRNA, B) constructs begins to degrade almost immediately as indicated by the observed ladder effect on the PAGE gel. Also, the modified siRNAs have less mobility on the PAGE gel than the non-modified siRNA duplex.

Thus, it has been unexpectedly and surprisingly discovered that siRNA stability in plasma is enhanced when uridines are replaced with 5-methyluridines (ribothymidines)

### EXAMPLE 4

### Non-Modified and Modified LC20 and LC13 siRNAs

Table 3 presents a list of modified and non-modified forms of LC13 siRNAs. Table 4 presents a list of modified and non-modified forms of LC20 siRNAs. The modified forms of these siRNAs include 2′-O-methyl modified ribonucleotides alone or in combination with substituting uridines with ribothymidines (5-methyluridine). A 2′-O-methyl modified ribonucleotide is indicated by a “MeO” above the ribonucleotide (e.g., N^{MeO} where N is the ribonucleotide). A ribothymidine is indicated by an “r” above the ribonucleotide (e.g., N^{r}). Each specific siRNA modification is assigned a particular label which is placed behind the LC20 and LC13 name. This allows for a direct comparison of siRNA stability and knockdown activity between two different siRNAs that have the same modification (e.g., LC13-Md15 has the same modification as LC20-MD15).

TABLE 3

| | | |
|---|---|---|
| siRNA | Nucleotide Sequence | Sequence ID |

| | | |
|---|---|---|
| LC13-WT | 5′- UCCUCAGCCUCUUCUCCUUdTdT - 3′ | SEQ ID NO: 23 |
| Non-modified | 3′- dTdTAGGAGUCGGAGAAGAGGAAp - 5′ | SEQ ID NO: 24 |
| LC13-19mer | 5′- UCCUCAGCCUCUUCUCCUU - 3′ | SEQ ID NO: 25 |
| No 3′ Overhangs | 3′- AGGAGUCGGAGAAGAGGAAp - 5′ | SEQ ID NO: 26 |
| LC13-Md3 | 5′- UCCUCAGCCUCUUCUCCU^{MeO}U^{MeO}dTdT - 3′ | SEQ ID NO: 27 |
| | 3′- dTdTA^{MeO}G^{MeO}GAGUCGGAGAAGAGGAAp - 5′ | SEQ ID NO: 28 |
| LC13-Md4 | 5′- U^{MeO}C^{MeO}CUCAGCCUCUUCUCCU^{MeO}U^{MeO}dTdT - 3′ | SEQ ID NO: 29 |
| | 3′- dTdTA^{MeO}G^{MeO}GAGUCGGAGAAGAGGA^{MeO}A^{MeO}p - 5′ | SEQ ID NO: 30 |
| LC13-Md5 | 5′- U^{MeO}C^{MeO}CUCAGCCUCUUCUCCUUdTdT - 3′ | SEQ ID NO: 31 |
| | 3′- dTdTAGGAGUCGGAGAAGAGGA^{MeO}A^{MeO}p - 5′ | SEQ ID NO: 32 |
| LC13-Md6 | 5′- T^{r}CCT^{r}CAGCCT^{r}CT^{r}CT^{r}CCU^{MeO}U^{MeO}dT dT - 3′ | SEQ ID NO: 33 |
| | 3′- dTdTA^{MeO}G^{MeO}GAGT^{r}CGGAGAAGAGGAA p - 5′ | SEQ ID NO: 34 |
| LC13-Md7 | 5′- U^{MeO}C^{MeO}CT^{r}CAGCCT^{r}CT^{r}T^{r}CT^{r}CCU^{MeO}U^{MeO}dTdT - 3′ | SEQ ID NO: 35 |
| | 3′- dTdTA^{MeO}G^{MeO}GAGT^{r}CGGAGAAGAGGA^{MeO}A^{MeO}p - 5′ | SEQ ID NO: 36 |
| LC13-Md8 | 5′- U^{MeO}C^{MeO}CT^{r}CAGCCT^{r}CT^{r}T^{r}CT^{r}CCT^{r}T^{r}dTdT - 3′ | SEQ ID NO: 37 |
| | 3′- dTdTAGGAGT^{r}CGGAGAAGAGGA^{MeO}A^{MeO}p - 5′ | SEQ ID NO: 38 |
| LC13-Md12 | 5′- UCCUCAGCCUCUUCUCCUU^{MeO}dT dT - 3′ | SEQ ID NO: 39 |
| | 3′- dTdTA^{MeO}GGAGUCGGAGAAGAGGA Ap - 5′ | SEQ ID NO: 40 |
| LC13-Md13 | 5′- U^{MeO}CCT^{r}CAGCCT^{r}CT^{r}T^{r}CT^{r}CCT^{r}T^{r}dT dT - 3′ | SEQ ID NO: 41 |
| | 3′- dTdTAGGAGT^{r}CGGAGAAGAGGAA^{MeO}-p - 5′ | SEQ ID NO: 42 |
| LC13-Md14 | 5′- U^{MeO}CCUCAGCCUCUUCUCC^{MeO}dT dT - 3′ | SEQ ID NO: 43 |
| | 3′- dTdTAGGAGUCGGAGAAGAGGAAp - 5′ | SEQ ID NO: 44 |
| LC13-Md15 | 5′- U^{MeO}C^{MeO}CUCAGCCUCUUCUCCU^{MeO}U^{MeO}dT dT - 3′ | SEQ ID NO: 45 |
| | 3′- dTdTAGGAGUCGGAGAAGAGGAAp - 5′ | SEQ ID NO: 46 |
| LC13-Md16 | 5′- U^{MeO}CCUCAGCCUCUUCUCCUUdT dT - 3′ | SEQ ID NO: 47 |
| | 3′- dTdTAGGAGUCGGAGAAGAGGAA^{MeO}p - 5′ | SEQ ID NO: 48 |

TABLE 4

| | | |
|---|---|---|
| siRNA | Nucleotide Sequence | Sequence ID |

| | | |
|---|---|---|
| LC20-WT | 5′- GGGUCGGAACCCAAGCUUA dTdT - 3′ | SEQ ID NO: 49 |
| Non-modified | 3′- dAdT CCCAGCCUUGGGUUCGAAU-p - 5′ | SEQ ID NO: 50 |
| LC20- 19mer | 5′- GGGUCGGAACCCAAGCUUA - 3′ | SEQ ID NO: 51 |
| No 3′ Overhangs | 3′- CCCAGCCUUGGGUUCGAAU-p - 5′ | SEQ ID NO: 52 |
| and Non- | | |
| modified | | |
| LC20- | 5′- G^{MeO}G^{MeO}GU^{MeO}C^{MeO}GGAAC^{MeO}C^{MeO}C^{MeO}AAGC^{MeO}U^{MeO}U^{MeO}A - 3′ | SEQ ID NO: 53 |
| siSTABLE | 3′- UsUsC^{F}C^{F}C^{F}AGC^{F}C^{F}U^{F}U^{F}GGGU^{F}U^{F}C^{F}GAAU^{F}-p - 5′ | SEQ ID NO: 54 |
| LC20-MD3 | 5′- GGGUCGGAACCCAAGCUU^{MeO}A^{MeO}dTdT - 3′ | SEQ ID NO: 55 |
| | 3′- dAdT C^{MeO}C^{MeO}CAGCCUUGGGUUCGAAU-p - 5′ | SEQ ID NO: 56 |
| LC20-MD5 | 5′- G^{MeO}G^{MeO}GUCGGAACCCAAGCUUA dTdT - 3′ | SEQ ID NO: 57 |
| | 3′- dAdT CCCAGCCUUGGGUUCGAA^{MeO}U^{MeO}-p - 5′ | SEQ ID NO: 58 |
| LC20-MD6 | 5′- GGGT^{r}CGGAACCCAAGCT^{r} U^{MeO}A^{MeO} dTdT - 3′ | SEQ ID NO: 59 |
| | 3′- dAdT C^{MeO}C^{MeO}CAGCCT^{r}T^{r}GGGT^{r}T^{r}CGAAT^{r}-p - 5′ | SEQ ID NO: 60 |
| LC20-MD8 | 5′- G^{MeO}G^{MeO}GT^{r}CGGAACCCAAGCT^{r}T^{r}A dTdT - 3′ | SEQ ID NO: 61 |
| | 3′- dAdT CCCAGCCT^{r}T^{r}GGGT^{r}T^{r}CGAA^{MeO}U^{MeO}-p - 5′ | SEQ ID NO: 62 |
| LC20-MD15 | 5′- G^{MeO}G^{MeO}GUCGGAACCCAAGCUUA dTdT - 3′ | SEQ ID NO: 63 |
| | 3′- dAdT CCCAGCCUUGGGUUCGAAU-p - 5′ | SEQ ID NO: 64 |
| LC20-MD17 | 5′- GGGUCGGAACCCAAGCUU A dTdT - 3′ | SEQ ID NO: 65 |
| | 3′- dAdT C^{MeO}C^{MeO}CAGCCUUGGGUUCGAA^{MeO}U^{MeO}-p - 5′ | SEQ ID NO: 66 |
| LC20-MD18 | 5′- G^{MeO}G^{MeO}GT^{r}CGGAACCCAAGCT^{r}U^{MeO}A^{MeO}dTdT -3 ′ | SEQ ID NO: 67 |
| | 3′- dAdT CCCAGCCT^{r}T^{r}GGGT^{r}T^{r}CGAT^{r}-p - 5′ | SEQ ID NO: 68 |
| LC20-MD19 | 5′- GGGT^{r}CGGAACCCAAGCT^{r}T^{r}A dTdT - 3′ | SEQ ID NO: 69 |
| | 3′- dAdT C^{MeO}C^{MeO} CAGCCT^{r}T^{r}GGGT^{r}T^{r}CGAA^{MeO}U^{MeO}-p - 5′ | SEQ ID NO: 70 |
| LC20-MD20 | 5′- GGGUCGGAACCCAAGCUU^{MeO}A^{MeO}dTdT - 3′ | SEQ ID NO: 71 |
| | 3′- dAdTCCCAGCCUUGGGUUCGAAU-p - 5′ | SEQ ID NO: 72 |
| LC20-MD21 | 5′- G^{MeO}G^{MeO}GT^{r}CGGAACCCAAGCT^{r}U^{MeO}A^{MeO}dTdT - 3′ | SEQ ID NO: 73 |
| | 3′- CCCAGCCUUGGGUUCGAAU-p - 5′ | SEQ ID NO: 74 |
| LC20-MD23 | 5′- GGGT^{r}CGGAACCCAAGCT^{r}U^{MeO}A^{MeO}dTdT - 3′ | SEQ ID NO: 75 |
| | 3′- CCCAGCCUUGGGUUCGAAU-p - 5′ | SEQ ID NO: 76 |

### EXAMPLE 5

### 2′-O-methyl Modified Ribonucleotides Improved siRNA Stability in Plasma

The purpose of this experiment was to determine whether 2′-O-methyl modified ribonucleotides and the substitution of uridines with ribothymidines (5-methyluridine) provide for greater siRNA stability against rat plasma ribonucleases. Improved stability was observed for both LC20 and LC13 indicating that these modifications will promote stability among all siRNAs universally. The siRNA duplexes listed in Table 3 and Table 4 in Example 4 were tested. The tables below show the stability rankings for the non-modified and modified forms of LC20 siRNA (Table 5) and LC13 siRNA (Table 6) whereby a stability ranking of one is most stable.

TABLE 5

| siRNA | Stability Ranking |
|---|---|
| LC20-MD15 | 1 |
| LC20-MD5 | |
| LC20-MD3 | |
| LC20-MD6 | |
| LC20-MD19 | |
| LC20-MD8 | |
| LC20-MD17 | 2 |
| LC20-MD18 | |
| LC20-MD20 | |
| LC20-MD21 | 3 |
| LC20-MD23 | |
| LC20-MD22 | 4 |
| LC20-WT | |
| Non-modified | |
| LC20-19mer | |
| No 3′ overhangs | |
| and Non- | |
| modified | |

TABLE 6

| siRNA | Stability Ranking |
|---|---|
| LC13-Md4 | 1 |
| LC13-Md8 | |
| LC13-Md15 | |
| LC13-Md3 | |
| LC13-Md6 | 2 |
| LC13-Md7 | |
| LC13-Md5 | |
| LC13-Md14 | 3 |
| LC13-Md12 | |
| LC13-Md13 | 4 |
| LC13-Md16 | |
| LC13- 19mer | |
| No 3′ overhangs | |
| and Non- | |
| modified | |
| LC20-WT Non- | |
| modified | |

A 20 μg aliquot of each siRNA duplex of Example 4 was mixed with 200 μl of fresh rat plasma incubated at 37° C. At various time points (0, 30, 60 and 20 min), 50 μl of the mixture was taken out and immediately extracted by phenol:chloroform. siRNAs were dried following precipitation by adding 2.5 volume of isopropanol alcohol and subsequent washing step with 70% ethanol. After resuspending in water and gel loading buffer the samples were analyzed gel electrophoresis on a 20% polyacrylamide gel, containing 7 M urea and subsequently visualized by ethidium bromide staining and quantified by densitometry.

Non-modified siRNA molecules were shown to be unstable in plasma. Surprisingly, however, siRNAs containing 2′ O-methyl modified ribonucleotides showed improved stability. More specifically, the greatest overall increase in LC20 siRNA stability was observed where two 2′O-methyl ribonucleotides were placed at the 5′-end and at the 3′-end, prior to the 3′ overhang, of the sense strand (LC20-MD15). Of note, the same modification to LC20 siRNA but in the anti-sense strand does not give the same degree of stability indicating that the stability enhancing effect of 2′O-methyl modified ribonucleotides may be strand specific. The greatest overall increase in LC13 siRNA stability was observed in the presence of two 2′-O-methyl ribonucleotides at the 5′-end and at the 3′-end, prior to the 3′ overhand, of both the sense and anti-sense strands (LC13-Md4). Thus, siRNA duplex stability improves with the increasing presence of 2′O-methyl modified ribonucleotides at or near the ends of the siRNA duplex. In general, these data show the surprisingly and unexpected discovery that siRNA duplex stability in plasma is improved in the presence of 2′ O-methyl modified ribonucleotides at or near the ends of the siRNA duplex.

In general, the replacement of uridines with ribothymidines in combination with 2′ O-methyl modified ribonucleotides did not significantly affect siRNA duplex stability.

### EXAMPLE 6

### Ribothymidines Improve siRNA Stability by Increasing the Melting Temperature (T_{M}) of the siRNA Duplex

The purpose of this experiment was to determine the effect of incorporating ribothymidines in a double stranded RNA molecule in place of uridines on the melting temperature (T_{M}) of the siRNA molecule. A higher Tₘ correlates with increased siRNA duplex stability.

To determine the effect of replacing uridines with ribothymidines in the siRNA duplex upon melting temperature, thermal melting profiles were generated for four β-galactosidase (β-gal) siRNA molecules (Table 7). These four β-gal siRNA duplexes differ only by the presence or absence of ribothymidines in the siRNA duplex. Where ᵣT is 5-methyluridine.

TABLE 7

| | | | |
|---|---|---|---|
| Duplex | Sequence | ID | Sequence ID |

| | | | |
|---|---|---|---|
| βgal-U (Homoduplex; | C U A C A C A A A U C A G C G A U U U TT | I | SEQ ID NO: 77 |
| WT) | TT G A U G U G U U U A G U C G C U A A A | | SEQ ID NO: 78 |
| βgal-T^{r} (Homoduplex) | C T^{r} A C A C A A A T^{r} C A G C G A T^{r} T^{r} T^{r}TT | II | SEQ ID NO: 79 |
| | TT G AT^{r} GT^{r} GT^{r}T^{r}T^{r} A GT^{r} C G C T^{r} A A A | | SEQ ID NO: 80 |
| βgal-U/βgal-T^{r} | C U A C A C A A A U C A G C G A U U U T T | III | SEQ ID NO: 81 |
| | TT G AT^{r} GT^{r} GT^{r}T^{r} T^{r} A GT^{r} C G C T^{r} A A A | | SEQ ID NO: 82 |
| βgal-T^{r}/βgal-U | CT^{r} A C A C A A AT^{r} C A G C G A T^{r}T^{r}T^{r} T T | IV | SEQ ID NO: 83 |
| | TT G A U G U G U U U A G U C G C U A A A | | SEQ ID NO: 84 |

The stock solutions of single stranded oligonucleotides were prepared by dissolving the selected sequences in 400 μL 10 mM buffer phosphate pH 7.0 containing 0.1 M NaCl and 0.1 mM EDTA and diluted (1 μL to 200 μL) with water and absorbencies (A₂₆₀) were measured and the contents were calculated. Also the integrity of the oligonucleotides was confirmed by HPLC analysis. To prepare the siRNA duplexes, the single stranded oligonucleotides were mixed and allowed to anneal. The UV absorption (A₂₆₀) for each siRNA duplex was measured and their values are as follows: I (0.28), II (0.54), III (0.31), and IV (0.45). To test for reproducibility, the melting profile study was done in duplicate.

The thermal melting profiles of the duplexes I, II, III and IV were recorded on Shimadzu UV-VIS 1601 with thermoelectrically temperature controlled through the Peltier device. The temperature was changed at the rate of 0.5° C./minute from 90° C. to 25° C. while the absorption recorded at 260 nm. The reverse experiment is also repeated. The “melting” process is a physical phenomenon. Therefore, the generated profile (90° to 25°) ought to be superimposed on the reverse (20° to 90°).

Differential curves were used to determine the melting point (Tₘ) of the duplexes. The shape of the curve defined by the derivative of a curve (versus 1/T) was used to make a robust determination of Tₘ and other thermodynamic data. Shimadzu TMSPC-8 and its associated software performed the needed calculations. The Tₘs have been listed in Table 8. The duplicate experiment (2^{nd} Experiment; table not shown) had a similar thermal melting profile.

TABLE 8

| Duplex (ID) | Up/Down profile | Tₘ (° C.) |
|---|---|---|
| I (WT) | Up | 64.3 |
| I (WT) | Down | 65.6 |
| II | Up | 71.9 |
| II | Down | 72.8 |
| III | Up | 71.9 |
| III | Down | 72.9 |
| IV | Up | 67.8 |
| IV | Down | 67.8 |

As shown in Table 9, a direct comparison was done between the duplicate experiments. The differences between the Tₘs derived from the 1^{st} Experiment and the 2^{nd} Experiment are shown in the far right column (ΔTₘ). Minimal to no difference was observed between the two experiments.

TABLE 9

| | Avg. Tₘ from 1^{st} Exp. | Avg. Tₘ from 2^{nd} Exp. | |
|---|---|---|---|
| Duplex | (° C.) | (° C.) | ΔTₘ |
| I (WT) | 64.9 | 64.9 | 0.0 |
| II | 72.35 | 72.25 | 0.1 |
| III | 72.4 | 69.8 | 2.6 |
| IV | 67.8 | 67.8 | 0.0 |

In conclusion, the incorporation of rT in the double stranded RNA in place of uridine residues increases the stability of the duplex by ˜0.6° C./rT incorporation. As shown in Table 9, duplex III (rT incorporated in the anti-sense strand only; a total of 7 rTs) melts at a temperature of approximately 4.9° higher than the wild type (duplex I). Duplex IV (rT incorporated in the sense stand only; a total of 5 rTs) melts at a temperature of 67.8° C., or 2.9° C. higher than the wild type. Finally, duplex II (rT incorporate in both the sense and anti-sense strands; 12 rTs) melts at about 72.3° C., or 7.4° C. higher than the wild type.

Thus, these data indicate the surprisingly and unexpected discovery that the Tₘ of the wild type β-gal RNA duplex is increased when the uridines are replaced with ribothymidines. Consequently, because of the increased Tₘ, the stability of the RNA duplex is increased.

### EXAMPLE 7

### siRNA Gene Knockdown Activity is Enhanced with 2′-O-methyl Ribonucleotides and Ribothymidines

The purpose of this experiment was to determine whether 2-′O-methyl modified ribonucleotides in combination with the substitution of uridines with ribothymidines in the siRNA duplex would enhance its ability to downregulate target gene expression. siRNA knockdown activity was determined with a similar protocol as described in Example 2 except that siRNAs were transfected with the polynucleotide delivery-enhancing polypeptide PN73. The amino acid sequence of the polynucleotide-delivery enhancing peptide PN73 is as follows: NH₂-KGSKKAVTKAQKKDGKKRKRSRKESYSVYVYKVLKQ (SEQ ID NO: 85). PN73 was mixed with each siRNA at a 1:5 ratio. Each siRNA was tested at a concentration of 0.16 nM, 0.8 nM and 4 nM.

Non-modified forms of LC20 and LC13 with 3′ overhangs (LC20-WT and LC13-WT) were used as a baseline to determine whether modified siRNAs had increased target gene knockdown activity compared to non-modified siRNAs. Also, a random siRNA sequence was used as a negative control (Qneg).

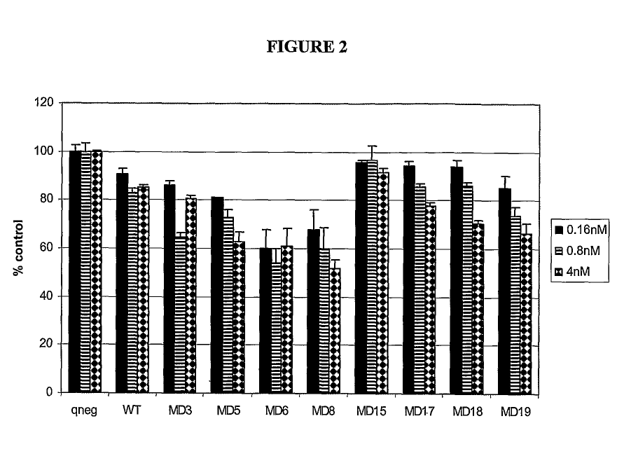

FIG. 2 shows the knockdown activities for LC20-MD3, MD-5, MD-6, MD-5, MD-15, MD-17, MD-18 and MD19. The solid bars represent an siRNA concentration of 0.16 nM, the bars with horizontal stripes represent an siRNA concentration of 0.8 nM and the bars with black and white diamonds represent an siRNA concentration of 4 nM. Knockdown activities were normalized to the Qneg control siRNA and presented as a percentage of the Qneg control (i.e., Qneg represented 100% or “normal” gene expression levels). Thus, a smaller percentage indicates a greater knockdown effect. As shown in FIG. 2, the negative control, Qneg, showed no measurable knockdown activity. The greatest overall knockdown activity for LC20 was observed when two 2′O-methyl modified ribonucleotides were placed at the 5′-end of both the sense and anti-sense strands and all remaining uridines were converted to ribothymidines (e.g., LC20-MD6 and LC20-MD8).

Knockdown activities for modified LC13 siRNAs were also measured. The greatest overall knockdown activity for LC13 was observed with LC13-Md13 and LC13-Md15. LC13-Md13 has one 2′O-methyl modified ribonucleotide at the 5′-end of each strand and the remaining uridines are replaced with ribothymidines. LC13-Md15 has two 2′O-methyl modified ribonucleotides at the 5′-end and 3′-end of the sense strand.

In addition, the knockdown activity of siSTABLE and non-modified siRNAs were compared. The non-modified siRNAs provided a greater knockdown activity compared to the same siRNAs in siSTABLE form. Thus, siSTABLE modification of siRNAs does not provide increased knockdown activity over the non-modified form. Furthermore, siSTABLE siRNAs with 2′O-methyl modified ribonucleotides and/or ribothymidine substitutions did not change siSTABLE siRNA activity.

In general, these data show the surprisingly and unexpected discovery that siRNA duplex knockdown activity can be improved with the addition of 2′ O-methyl modified ribonucleotides at or near the ends of the siRNA duplex and where ribothymidines are substituted for uridines within the siRNA molecule.

### EXAMPLE 8

### siRNA Off Target Effect is Minimized with 2′-O-methyl Ribonucleotides and Ribothymidines

The purpose of this experiment was to determine whether siRNA gene target specificity could be enhanced with 2′-O-methyl modified ribonucleotides and ribothymidine substitutions in the siRNA duplex. Although siRNA is a powerful technique used to disrupt the expression of target genes, an undesired consequence of this method is that it may also effect the expression of non-target genes (off-target effect). Thus, to determine if the off-target effect of siRNA molecules could be minimized with the addition of 2′-O-methyl ribonucleotides and ribothymidines, an off-target profile was generated for 5 different siRNAs that target the human tumor necrosis factor-α (TNF-α) mRNA. The modified siRNA was based on the MD8 modification listed in Tables 3 and 4 of Example 4. An example of an LC20-siSTABLEv2 modified siRNA is shown as Table 4 of Example 4.

Agilent microarrays were used and consisted of 60-mer probe oligonucleotides (targets) representing ˜18,500 well-characterized, full-length human genes. The non-modified siRNA candidates showed an off-target effect of between 5 to 84 gene expression changes out of a total of 18,500 genes. An off-target gene effect was counted when a 2-fold change (up or down) in gene expression was observed. The siSTABLEv2 modified siRNAs showed a decreased off-target effect. Surprisingly, the siRNA candidates with the full ribothymidine substitution, 3′-ends with 2 base dT overhangs, and 5′-end dinucleotide 2′-O-methyl substituted riboses showed minimal off-target effects (Table 10). In particular, TNFα-2, TNFα-17 and LC20 siRNAs with 2′O-methyl modified ribonucleotides and ribothymidines showed no off-target effect.

TABLE 10

| | Non-modified | | Modified siRNA |
|---|---|---|---|
| siRNA | siRNA Off- | siSTABLEv2 | Off-Target Effect |
| Candidate | Target Effect | Modified | (2′-O-methyl + riboT) |
| TNFα-2 | 33 | 2 | 0 |
| TNFα-9 | 69 | 3 | 4 |
| TNFα-17 | 84 | 2 | 0 |
| LC17 | 51 | 9 | 12 |
| LC20 | 5 | 3 | 0 |

The siRNA modification had a significant effect on reducing off-target responses. From this data, the extent of G:U base pairing in all the identified siRNA off-target interactions should be able to be evaluated and therefore, the potential ribothymidine substitutions needed to eliminate the off-target effects by the suppression of G:U wobble should be able to be ascertained.

These data show the surprisingly and unexpected discovery that the siRNA off target effect can be minimized or even ablated by the addition of 2′-O-methyl modified ribonucleotides and ribothymidine substitutions.

### EXAMPLE 9

### Interferon Response

Interferon responsiveness is a potential side-effect of transfecting cells with siRNAs. Thus, a study was performed in vitro to assess whether various isoforms of the LC20 siRNA would elicit an interferon response. Both non-modified and modified of the 19-mer LC20 siRNA and 21-mer LC20 siRNA were tested. The 21-mer LC20 siRNA contains 2 base pair 5′-end overhangs while the 19-mer LC20 siRNA does not.

The non-modified 21-mer and 19-mer forms of the LC20 siRNA did not elicit an interferon response. Furthermore, the 21-mer LC20-MD8 modified siRNA, which includes two 2′O-methyl modified ribonucleotides at the 5′-end of each strand and the replacement of all remaining non-modified uridines with ribothymidines, did not elicit an interferon response. However, the identical modification of LC20 but in the 19-mer length induced an interferon response.

### EXAMPLE 10

### Stability of Universal-binding Nucleotide Comprising siRNA in Rat Plasma

This Example discloses a suitable animal model system for determining the in vivo stability of a universal-binding nucleotide comprising siRNA of the present invention.

A 20 μg aliquot of each universal-binding nucleotide comprising siRNA duplex of are mixed with 200 μl of fresh rat plasma incubated at 37° C. At various time points (0, 30, 60 and 20 min), 50 μl of the mixture are taken out and immediately extracted by phenol:chloroform. SiRNAs are dried following precipitation by adding 2.5 volumes of isopropanol alcohol and subsequent washing step with 70% ethanol. After dissolving in water and gel loading buffer the samples are analyzed on 20% polyacrylamide gel, containing 7 M urea and visualized by ethidium bromide staining and quantitated by densitometry. The level of degradation at each time point may be assessed by electrophoresis on a PAGE gel.

### EXAMPLE 11

### Measurement of Gene Knockdown Activity by Universal-Binding Nucleotide Comprising siRNA

This Example discloses suitable methodology for determining whether universal-binding nucleotide comprising siRNA of the present invention are capable of enhancing the ability of the siRNA to downregulate expression of one or more target genes.

SiRNA knockdown activity is determined by transfecting siRNAs with the polynucleotide delivery-enhancing polypeptide PN73. PN73 is mixed with each siRNA at a 1:5 ratio. Each siRNA is tested at a concentration of 0.16 nM, 0.8 nM, and 4 nM. Non-modified forms of siRNA are used as a nucleotide line to determine whether modified siRNAs exhibit increased variant target gene knockdown activity as compared to non-modified siRNAs. A random siRNA sequence may be used as a negative control.

### EXAMPLE 12

### Measurement of Off Target Effect by Universal-Binding Nucleotide Comprising siRNA

This Example provides a suitable methodology for measuring off-target effects mediated by universal-binding nucleotide comprising siRNA of the present invention.

Although siRNA of the present invention may be suitably employed for disrupting the expression of variant target genes, there remains the possibility that such siRNA may affect the expression of one or more non-target gene(s). Thus, an off-target profile may be generated for siRNAs that target a variant of an otherwise wild-type gene, such as a viral gene or an endogenous gene. Agilent microarrays may be employed that consist of 60-mer probe oligonucleotide targets representing, for example, ˜18,500 well-characterized, full-length human genes.

It is expected that siRNA modifications will have a significant effect on reducing off-target responses. The extent of G:U nucleotide pairing in all the identified siRNA off-target interactions are evaluated and, therefore, the potential of universal-binding nucleotides to eliminate the off-target effects by the suppression of G:U wobble may be ascertained.

### EXAMPLE 13

### Stability of Modified siRNA in Human Plasma

The present example demonstrates that the addition of a 2′-O-methoxy moiety to the ribose of the two most 5′-end nucleotides of both the sense and anti-sense strands of a siRNA duplex and the substitution of the remaining non-modified uridines of both the sense and anti-sense strands of the siRNA duplex with ribothymidines reduces the susceptibility of the siRNA duplex to degradation by nucleases present in human plasma. Specifically, the present example compares the degradation profiles between the non-modified and modified siRNA duplex after incubation with human plasma. The degradation profile includes both the degree of degradation over-time (degradation time-course) and the identity of the degradation products (“degradants”).

Both non-modified (LC20WT) and modified (LC20-MD8) siRNA include two deoxyribonucleotides at the 3′-end and a 5′-phosphate. The modified siRNA includes the addition of a 2′-O-methoxy moiety to the ribose of the two most 5′-end nucleotides of both the sense and anti-sense strands of the siRNA duplex and the substitution of the remaining non-modified uridines of both the sense and anti-sense strands of the siRNA duplex with ribothymidines (see Table 9). The non-modified and modified siRNA was incubated with 67% (final concentration) human plasma with lithium heparin (Bioreclamation, Inc.) for one minute, 60 minutes or 240 minutes at 37° C. Non-incubated non-modified and modified siRNA served as controls. After incubation with human plasma, siRNA was extracted with a phenol/chloroform kit (Trizol, Invitrogen) and characterized by dual detection HPLC. The HPLC system employed both a photodiode array (PDA) and mass selective detection (MS). MS was performed in single quad negative ion mode (Waters, Alliance 2695, Micromass ZQ). The column used was a XTerra column (Waters Corp.) and the following parameters were applied: MS C18, 2.1×50 mm, 2.5 μm held at 65° C. The mobile phase A used 100 mM hexafluoroisopropanol (HFIP) with 7 mM triethylamine (TEA), pH8.1 and phase B used methanol. The MS parameters were as follows: cap. 3.0 kV, cone −45V, desolv. 300° C. and 600 L/hr (N2), source temperature 90° C., 1000-2000 m/z over 1 s.

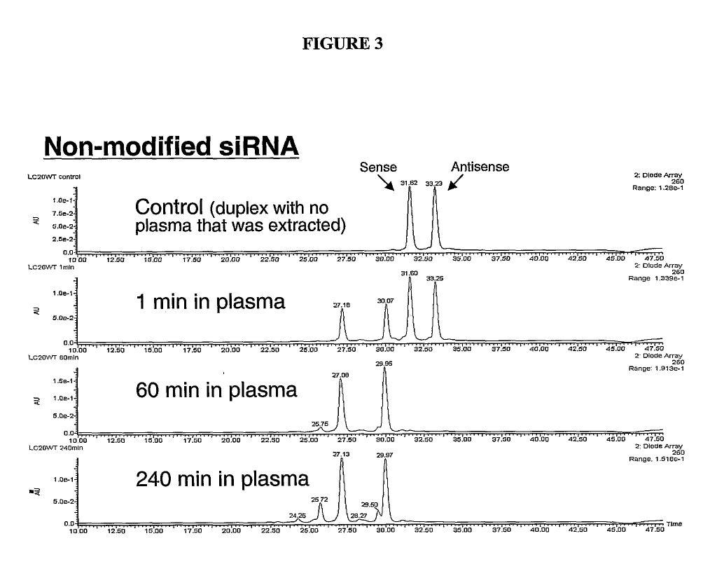

FIG. 3 shows the degradation time-course and the degradants for the non-modified siRNA duplex at time zero (non-incubated) and incubated with human plasma for 1 minute, 60 minutes and 240 minutes. As shown in FIG. 3, the sense strand of the non-modified siRNA duplex degraded at a faster rate and into more products than the anti-sense strand in human plasma. The exonuclease present in the human plasma was not deterred by the 5′ phosphate.

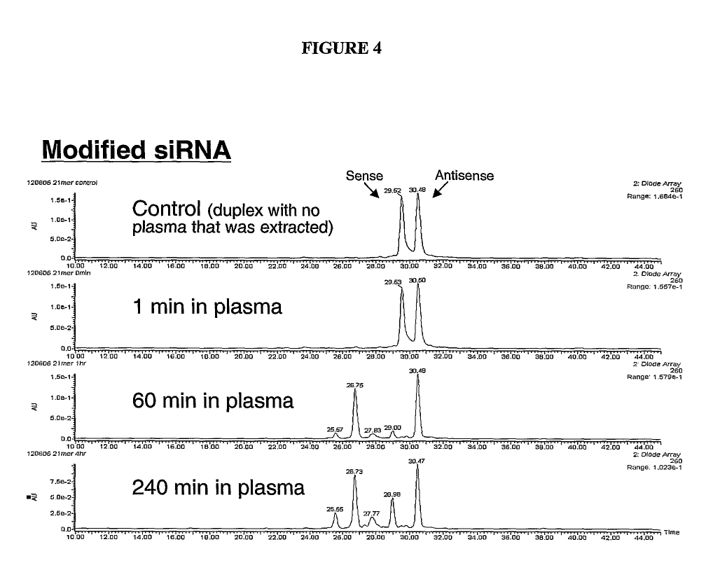

FIG. 4 shows the degradation time-course and the degradants for the modified siRNA duplex at time zero (non-incubated) and incubated with human plasma for 1 minute, 60 minutes and 240 minutes. As shown in FIG. 4, the sense strand of the modified siRNA exhibited a similar degradation pattern to that of the sense strand of the non-modified siRNA (see FIG. 3). However, the rate of degradation for the sense strand of the modified siRNA was slower than that of the sense strand of the non-modified siRNA as shown by delay in degradants for the sense strand of the modified siRNA compared to the sense strand of the non-modified siRNA (compare FIGS. 3 and 4). The slower degradation rates are likely due to the riboT modifications within the siRNA duplex.

The anti-sense strand of the modified siRNA duplex was cleaved from the 3′ terminus, in contrast to the anti-sense strand of the non-modified siRNA that was cleaved from the 5′ terminus.

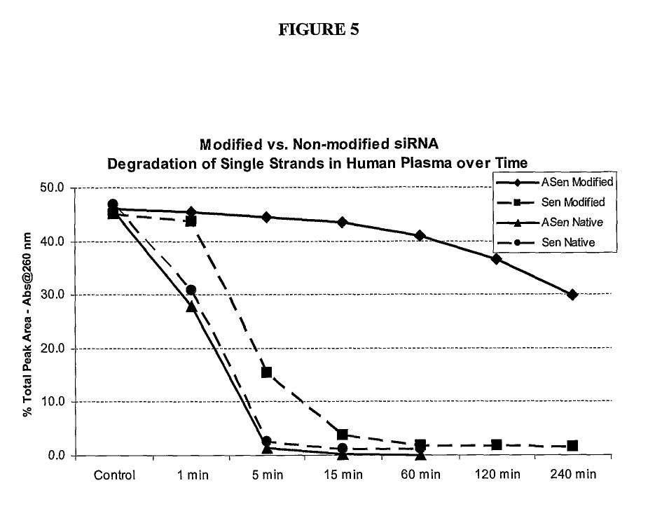

The degradation profiles for both the sense and anti-sense strands of the non-modified (native) and modified siRNA duplexes are summarized in FIG. 5.

These data show that the addition of the 2′-O-methoxy modification to the siRNA duplex significantly reduces the exonuclease activity found in human plasma.

In a related study, the modified siRNA was incubated in 3% fetal bovine serum (FBS) with and without lipofectamine. FBS is used in knockdown assays to sustain cell viability and lipofectamine is a common transfection agent. A nearly identical degradation profile was observed for FBS both with and without lipofectamine implying that the nucleases in FBS are similar to those in human plasma and lipofectamine binding to the siRNA does not offer any additional stability.

The teachings of all of references cited herein including patents, patent applications and journal articles are incorporated herein in their entirety by reference. Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. It is noted, however, that the various publications discussed herein are incorporated solely for their disclosure prior to the filing date of the present application, and the inventors reserve the right to antedate such disclosure by virtue of prior invention.

1.-112. (canceled)

113. A double-stranded RNA (dsRNA) molecule, comprising from 15 to 40 base pairs, and one or more multiply-modified ribonucleotide according to Formula I: 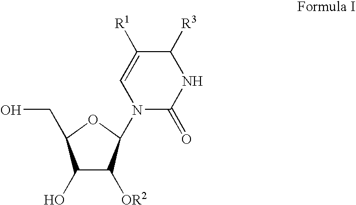
wherein R¹ and R² are each independently a halogen, hydroxy, alkyl, alkoxy, nitro, amino, trifluoromethyl, alkyl, cycloalkyl, (cycloalkyl)alkyl, alkanoyl, alkanoyloxy, aryl, aroyl, aralkyl, nitrile, dialkylamino, alkenyl, alkynyl, hydroxyalkyl, aminoalkyl, alkylaminoalkyl, dialkylaminoalkyl, haloalkyl, carboxyalkyl, alkoxyalkyl, carboxy, alkanoylamino, carbamoyl, carbamyl, carbonylamino, alkylsulfonylamino, or heterocyclo group; and
and R³ is an oxygen or an amino group.

114. The dsRNA molecule of claim 113 wherein R¹ is an alkyl group.

115. The dsRNA molecule of claim 113 wherein R¹ is methyl.

116. The dsRNA molecule of claim 113 wherein R² is an alkyl group.

117. The dsRNA molecule of claim 113 wherein R² is methyl.

118. The dsRNA molecule of claim 113 wherein R¹ is an alkyl group and R² is an alkyl group.

119. The dsRNA molecule of claim 113 wherein R¹ and R² are each a methyl.

120. The dsRNA molecule according to claim 1 wherein the dsRNA molecule comprises a sense strand of 25 or fewer nucleotides, and wherein the antisense strand has from 18 to 25 nucleotides.

121. The dsRNA molecule of claim 113 wherein said siRNA molecule further has one or more 3′-overhangs.

122. The dsRNA molecule of claim 113 comprising two or more of the multiply-modified ribonucleotides.

123. The dsRNA molecule of claim 113 comprising four or more of the multiply-modified ribonucleotides.

124. A composition for preventing or treating a disorder associated with overexpression of a disease-associated protein, or which is amenable to treatment by targeted reduction of expression of a disease-associated protein, in an animal subject comprising administering an effective amount of a dsRNA molecule of claim 113.

125. A composition of claim 124 wherein said disorder is selected from the group consisting of a cellular proliferative disorder, a differentiative disorder, a bone metabolic disorder, an immune disorder, an hematopoietic disorder, a cardiovascular disorder, a liver disorder, and a viral disease.

126. A method for reducing the expression of a target endogenous gene, comprising administering a dsRNA molecule of claim 113 to a cell expressing the endogenous target gene, wherein the dsRNA molecule is capable of specifically binding to the endogenous target gene thereby reducing its expression level in the cell.


SEQUENCE LISTING (88 sequences)

SEQ ID NO: 1 - DNA, 21 bp, Artificial Sequence
  Description of Combined DNA/RNA Molecule Synthetic oligonucleotide
cnacacaaancagcgannntt

SEQ ID NO: 2 - DNA, 21 bp, Artificial Sequence
  Description of Combined DNA/RNA Molecule Synthetic oligonucleotide
aaancgcngannngngnagtt

SEQ ID NO: 3 - DNA, 21 bp, Artificial Sequence
  Description of Combined DNA/RNA Molecule Synthetic oligonucleotide
cnacacaaancagcgannntt

SEQ ID NO: 4 - DNA, 21 bp, Artificial Sequence
  Description of Combined DNA/RNA Molecule Synthetic oligonucleotide
aaaucgcugauuuguguagtt

SEQ ID NO: 5 - DNA, 21 bp, Artificial Sequence
  Description of Combined DNA/RNA Molecule Synthetic oligonucleotide
cuacacaaaucagcgauuutt

SEQ ID NO: 6 - DNA, 21 bp, Artificial Sequence
  Description of Combined DNA/RNA Molecule Synthetic oligonucleotide
aaancgcngannngngnagtt

SEQ ID NO: 7 - DNA, 21 bp, Artificial Sequence
  Description of Combined DNA/RNA Molecule Synthetic oligonucleotide
cuacacaaaucagcgauuutt

SEQ ID NO: 8 - DNA, 21 bp, Artificial Sequence
  Description of Combined DNA/RNA Molecule Synthetic oligonucleotide
aaaucgcugauuuguguagtt

SEQ ID NO: 9 - DNA, 20 bp, Artificial Sequence
  Description of Combined DNA/RNA Molecule Synthetic oligonucleotide
gcannnggcanaagaaantt

SEQ ID NO: 10 - DNA, 20 bp, Artificial Sequence
  Description of Combined DNA/RNA Molecule Synthetic oligonucleotide
annnncnnangccaaanctt

SEQ ID NO: 11 - DNA, 21 bp, Artificial Sequence
  Description of Combined DNA/RNA Molecule Synthetic oligonucleotide
ccngcngcnangccncanctt

SEQ ID NO: 12 - DNA, 21 bp, Artificial Sequence
  Description of Combined DNA/RNA Molecule Synthetic oligonucleotide
gangaggcanagcagcaggtt

SEQ ID NO: 13 - DNA, 21 bp, Artificial Sequence
  Description of Combined DNA/RNA Molecule Synthetic oligonucleotide
nnnggaaaggaccagcaaatt

SEQ ID NO: 14 - DNA, 21 bp, Artificial Sequence
  Description of Combined DNA/RNA Molecule Synthetic oligonucleotide
nnngcnggnccnnnccaaatt

SEQ ID NO: 15 - DNA, 21 bp, Artificial Sequence
  Description of Combined DNA/RNA Molecule Synthetic oligonucleotide
cacccngacaagcngccagtt

SEQ ID NO: 16 - DNA, 21 bp, Artificial Sequence
  Description of Combined DNA/RNA Molecule Synthetic oligonucleotide
cnggcagcnngncagggngtt

SEQ ID NO: 17 - DNA, 21 bp, Artificial Sequence
  Description of Combined DNA/RNA Molecule Synthetic oligonucleotide
ngcacnnnggagngancggtt

SEQ ID NO: 18 - DNA, 21 bp, Artificial Sequence
  Description of Combined DNA/RNA Molecule Synthetic oligonucleotide
ccgancacnccaaagngcatt

SEQ ID NO: 19 - DNA, 21 bp, Artificial Sequence
  Description of Combined DNA/RNA Molecule Synthetic oligonucleotide
gagncccgggaagccccagtt

SEQ ID NO: 20 - DNA, 21 bp, Artificial Sequence
  Description of Combined DNA/RNA Molecule Synthetic oligonucleotide
cnggggcnncccgggacnctt

SEQ ID NO: 21 - DNA, 21 bp, Artificial Sequence
  Description of Combined DNA/RNA Molecule Synthetic oligonucleotide
aaaggaaccnacnngnacatt

SEQ ID NO: 22 - DNA, 21 bp, Artificial Sequence
  Description of Combined DNA/RNA Molecule Synthetic oligonucleotide
ngnacaagnaggnnccnnntt

SEQ ID NO: 23 - DNA, 21 bp, Artificial Sequence
  Description of Combined DNA/RNA Molecule Synthetic oligonucleotide
uccucagccucuucuccuutt

SEQ ID NO: 24 - DNA, 21 bp, Artificial Sequence
  Description of Combined DNA/RNA Molecule Synthetic oligonucleotide
aaggagaagaggcugaggatt

SEQ ID NO: 25 - DNA, 19 bp, Artificial Sequence
  Description of Combined DNA/RNA Molecule Synthetic oligonucleotide
uccucagccucuucuccuu

SEQ ID NO: 26 - DNA, 19 bp, Artificial Sequence
  Description of Combined DNA/RNA Molecule Synthetic oligonucleotide
aaggagaagaggcugagga

SEQ ID NO: 27 - DNA, 21 bp, Artificial Sequence
  Description of Combined DNA/RNA Molecule Synthetic oligonucleotide
uccucagccucuucuccuutt

SEQ ID NO: 28 - DNA, 21 bp, Artificial Sequence
  Description of Combined DNA/RNA Molecule Synthetic oligonucleotide
aaggagaagaggcugaggatt

SEQ ID NO: 29 - DNA, 21 bp, Artificial Sequence
  Description of Combined DNA/RNA Molecule Synthetic oligonucleotide
uccucagccucuucuccuutt

SEQ ID NO: 30 - DNA, 21 bp, Artificial Sequence
  Description of Combined DNA/RNA Molecule Synthetic oligonucleotide
aaggagaagaggcugaggatt

SEQ ID NO: 31 - DNA, 21 bp, Artificial Sequence
  Description of Combined DNA/RNA Molecule Synthetic oligonucleotide
uccucagccucuucuccuutt

SEQ ID NO: 32 - DNA, 21 bp, Artificial Sequence
  Description of Combined DNA/RNA Molecule Synthetic oligonucleotide
aaggagaagaggcugaggatt

SEQ ID NO: 33 - DNA, 21 bp, Artificial Sequence
  Description of Combined DNA/RNA Molecule Synthetic oligonucleotide
nccncagccncnncnccuutt

SEQ ID NO: 34 - DNA, 21 bp, Artificial Sequence
  Description of Combined DNA/RNA Molecule Synthetic oligonucleotide
aaggagaagaggcngaggatt

SEQ ID NO: 35 - DNA, 21 bp, Artificial Sequence
  Description of Combined DNA/RNA Molecule Synthetic oligonucleotide
uccncagccncnncnccuutt

SEQ ID NO: 36 - DNA, 21 bp, Artificial Sequence
  Description of Combined DNA/RNA Molecule Synthetic oligonucleotide
aaggagaagaggcngaggatt

SEQ ID NO: 37 - DNA, 21 bp, Artificial Sequence
  Description of Combined DNA/RNA Molecule Synthetic oligonucleotide
uccncagccncnncnccnntt

SEQ ID NO: 38 - DNA, 21 bp, Artificial Sequence
  Description of Combined DNA/RNA Molecule Synthetic oligonucleotide
aaggagaagaggcngaggatt

SEQ ID NO: 39 - DNA, 21 bp, Artificial Sequence
  Description of Combined DNA/RNA Molecule Synthetic oligonucleotide
uccucagccucuucuccuutt

SEQ ID NO: 40 - DNA, 21 bp, Artificial Sequence
  Description of Combined DNA/RNA Molecule Synthetic oligonucleotide
aaggagaagaggcugaggatt

SEQ ID NO: 41 - DNA, 21 bp, Artificial Sequence
  Description of Combined DNA/RNA Molecule Synthetic oligonucleotide
uccncagccncnncnccnntt

SEQ ID NO: 42 - DNA, 21 bp, Artificial Sequence
  Description of Combined DNA/RNA Molecule Synthetic oligonucleotide
aaggagaagaggcngaggatt

SEQ ID NO: 43 - DNA, 21 bp, Artificial Sequence
  Description of Combined DNA/RNA Molecule Synthetic oligonucleotide
uccucagccucuucuccuutt

SEQ ID NO: 44 - DNA, 21 bp, Artificial Sequence
  Description of Combined DNA/RNA Molecule Synthetic oligonucleotide
aaggagaagaggcugaggatt

SEQ ID NO: 45 - DNA, 21 bp, Artificial Sequence
  Description of Combined DNA/RNA Molecule Synthetic oligonucleotide
uccucagccucuucuccuutt

SEQ ID NO: 46 - DNA, 21 bp, Artificial Sequence
  Description of Combined DNA/RNA Molecule Synthetic oligonucleotide
aaggagaagaggcugaggatt

SEQ ID NO: 47 - DNA, 21 bp, Artificial Sequence
  Description of Combined DNA/RNA Molecule Synthetic oligonucleotide
uccucagccucuucuccuutt

SEQ ID NO: 48 - DNA, 21 bp, Artificial Sequence
  Description of Combined DNA/RNA Molecule Synthetic oligonucleotide
aaggagaagaggcugaggatt

SEQ ID NO: 49 - DNA, 21 bp, Artificial Sequence
  Description of Combined DNA/RNA Molecule Synthetic oligonucleotide
gggucggaacccaagcuuatt

SEQ ID NO: 50 - DNA, 21 bp, Artificial Sequence
  Description of Combined DNA/RNA Molecule Synthetic oligonucleotide
uaagcuuggguuccgacccta

SEQ ID NO: 51 - DNA, 19 bp, Artificial Sequence
  Description of Combined DNA/RNA Molecule Synthetic oligonucleotide
gggucggaacccaagcuua

SEQ ID NO: 52 - DNA, 19 bp, Artificial Sequence
  Description of Combined DNA/RNA Molecule Synthetic oligonucleotide
uaagcuuggguuccgaccc

SEQ ID NO: 53 - DNA, 19 bp, Artificial Sequence
  Description of Combined DNA/RNA Molecule Synthetic oligonucleotide
gggucggaacccaagcuua

SEQ ID NO: 54 - DNA, 21 bp, Artificial Sequence
  Description of Combined DNA/RNA Molecule Synthetic oligonucleotide
uaagcuuggguuccgacccuu

SEQ ID NO: 55 - DNA, 21 bp, Artificial Sequence
  Description of Combined DNA/RNA Molecule Synthetic oligonucleotide
gggucggaacccaagcuuatt

SEQ ID NO: 56 - DNA, 21 bp, Artificial Sequence
  Description of Combined DNA/RNA Molecule Synthetic oligonucleotide
uaagcuuggguuccgacccta

SEQ ID NO: 57 - DNA, 21 bp, Artificial Sequence
  Description of Combined DNA/RNA Molecule Synthetic oligonucleotide
gggucggaacccaagcuuatt

SEQ ID NO: 58 - DNA, 21 bp, Artificial Sequence
  Description of Combined DNA/RNA Molecule Synthetic oligonucleotide
uaagcuuggguuccgacccta

SEQ ID NO: 59 - DNA, 21 bp, Artificial Sequence
  Description of Combined DNA/RNA Molecule Synthetic oligonucleotide
gggncggaacccaagcnuatt

SEQ ID NO: 60 - DNA, 21 bp, Artificial Sequence
  Description of Combined DNA/RNA Molecule Synthetic oligonucleotide
naagcnngggnnccgacccta

SEQ ID NO: 61 - DNA, 21 bp, Artificial Sequence
  Description of Combined DNA/RNA Molecule Synthetic oligonucleotide
gggncggaacccaagcnnatt

SEQ ID NO: 62 - DNA, 21 bp, Artificial Sequence
  Description of Combined DNA/RNA Molecule Synthetic oligonucleotide
uaagcnngggnnccgacccta

SEQ ID NO: 63 - DNA, 21 bp, Artificial Sequence
  Description of Combined DNA/RNA Molecule Synthetic oligonucleotide
gggucggaacccaagcuuatt

SEQ ID NO: 64 - DNA, 21 bp, Artificial Sequence
  Description of Combined DNA/RNA Molecule Synthetic oligonucleotide
uaagcuuggguuccgacccta

SEQ ID NO: 65 - DNA, 21 bp, Artificial Sequence
  Description of Combined DNA/RNA Molecule Synthetic oligonucleotide
gggucggaacccaagcuuatt

SEQ ID NO: 66 - DNA, 21 bp, Artificial Sequence
  Description of Combined DNA/RNA Molecule Synthetic oligonucleotide
uaagcuuggguuccgacccta

SEQ ID NO: 67 - DNA, 21 bp, Artificial Sequence
  Description of Combined DNA/RNA Molecule Synthetic oligonucleotide
gggncggaacccaagcnuatt

SEQ ID NO: 68 - DNA, 20 bp, Artificial Sequence
  Description of Combined DNA/RNA Molecule Synthetic oligonucleotide
nagcnngggnnccgacccta

SEQ ID NO: 69 - DNA, 21 bp, Artificial Sequence
  Description of Combined DNA/RNA Molecule Synthetic oligonucleotide
gggncggaacccaagcnnatt

SEQ ID NO: 70 - DNA, 21 bp, Artificial Sequence
  Description of Combined DNA/RNA Molecule Synthetic oligonucleotide
uaagcnngggnnccgacccta

SEQ ID NO: 71 - DNA, 21 bp, Artificial Sequence
  Description of Combined DNA/RNA Molecule Synthetic oligonucleotide
gggucggaacccaagcuuatt

SEQ ID NO: 72 - DNA, 21 bp, Artificial Sequence
  Description of Combined DNA/RNA Molecule Synthetic oligonucleotide
uaagcuuggguuccgacccta

SEQ ID NO: 73 - DNA, 21 bp, Artificial Sequence
  Description of Combined DNA/RNA Molecule Synthetic oligonucleotide
gggncggaacccaagcnuatt

SEQ ID NO: 74 - DNA, 19 bp, Artificial Sequence
  Description of Combined DNA/RNA Molecule Synthetic oligonucleotide
uaagcuuggguuccgaccc

SEQ ID NO: 75 - DNA, 21 bp, Artificial Sequence
  Description of Combined DNA/RNA Molecule Synthetic oligonucleotide
gggncggaacccaagcnuatt

SEQ ID NO: 76 - DNA, 19 bp, Artificial Sequence
  Description of Combined DNA/RNA Molecule Synthetic oligonucleotide
uaagcuuggguuccgaccc

SEQ ID NO: 77 - DNA, 21 bp, Artificial Sequence
  Description of Combined DNA/RNA Molecule Synthetic oligonucleotide
cuacacaaaucagcgauuutt

SEQ ID NO: 78 - DNA, 21 bp, Artificial Sequence
  Description of Combined DNA/RNA Molecule Synthetic oligonucleotide
aaaucgcugauuuguguagtt

SEQ ID NO: 79 - DNA, 21 bp, Artificial Sequence
  Description of Combined DNA/RNA Molecule Synthetic oligonucleotide
cnacacaaancagcgannntt

SEQ ID NO: 80 - DNA, 21 bp, Artificial Sequence
  Description of Combined DNA/RNA Molecule Synthetic oligonucleotide
aaancgcngannngngnagtt

SEQ ID NO: 81 - DNA, 21 bp, Artificial Sequence
  Description of Combined DNA/RNA Molecule Synthetic oligonucleotide
cuacacaaaucagcgauuutt

SEQ ID NO: 82 - DNA, 21 bp, Artificial Sequence
  Description of Combined DNA/RNA Molecule Synthetic oligonucleotide
aaancgcngannngngnagtt

SEQ ID NO: 83 - DNA, 21 bp, Artificial Sequence
  Description of Combined DNA/RNA Molecule Synthetic oligonucleotide
cnacacaaancagcgannntt

SEQ ID NO: 84 - DNA, 21 bp, Artificial Sequence
  Description of Combined DNA/RNA Molecule Synthetic oligonucleotide
aaaucgcugauuuguguagtt

SEQ ID NO: 85 - protein, 36 aa, Artificial Sequence
  Description of Artificial Sequence Synthetic peptide
KGSKKAVTKAQKKDGKKRKRSRKESYSVYVYKVLKQ

SEQ ID NO: 86 - DNA, 13 bp, Artificial Sequence
  Description of Artificial Sequence Synthetic oligonucleotide
ggaaaanaaaagg

SEQ ID NO: 87 - DNA, 13 bp, Artificial Sequence
  Description of Artificial Sequence Synthetic oligonucleotide
ccttttnttttcc

SEQ ID NO: 88 - DNA, 12 bp, Artificial Sequence
  Description of Artificial Sequence Synthetic oligonucleotide
gggaannttccc